(19) United States
(12) Patent Application Publication
(10) Pub. No.: US 2019/0076064 A1
(43) Pub. Date: Mar. 14, 2019

(54) BLOOD PRESSURE MEASUREMENT STATE DETERMINATION METHOD, BLOOD PRESSURE MEASUREMENT STATE DETERMINING DEVICE, AND RECORDING MEDIUM

(71) Applicant: Panasonic Intellectual Property Corporation of America, Torrance, CA (US)

(72) Inventors: KOHEI TAHARA, Osaka (JP); SHOICHI IIZUKA, Osaka (JP); TAKASHI MORIMOTO, Tokyo (JP); TOMOKI OGAWA, Osaka (JP); SHIGENORI MAEDA, Shiga (JP); NOBUHIRO AKIMOTO, Shiga (JP)

(21) Appl. No.: 16/185,214

(22) Filed: Nov. 9, 2018

Related U.S. Application Data
(63) Continuation of application No. PCT/JP2017/026628, filed on Jul. 24, 2017.

(30) Foreign Application Priority Data
Jul. 28, 2016 (JP) 2016-148265
Aug. 1, 2016 (JP) 2016-151310
Jul. 14, 2017 (JP) 2017-137633

(51) Int. Cl. A61B 5/1171; A61B 5/00; G06K 9/00; A61B 5/022; G10L 25/51; G06Q 40/08

(52) U.S. Cl. CPC A61B 5/1176; A61B 5/7475; A61B 5/7435; G06K 2009/00328; A61B 5/022; G10L 25/51; G06Q 40/08; G06K 9/00288

(57) ABSTRACT

A method for determining a blood pressure measurement state, using a device that is held in a hand of a user to whom a blood pressure meter is mounted. The method includes: obtaining (i) image data including a face of the user by a camera that the device has, (ii) first information indicating an inclination angle of the determining device as to the gravitational direction, by an angle sensor that the device has, and (iii) second information indicating a position of the face of the user in the image data, and the proportion of the size of the face of the user in the image data; determining whether or not the user is correctly using the blood pressure meter based on the image data, the first information and the second information; and providing a notification indicating the determination result.

### BACKGROUND

### 1. Technical Field

The present disclosure relates to a technology for identifying a measurement subject of a measurement device that measures biometric information, and determining a blood pressure measurement state.

### 2. Description of the Related Art

Insurance products called risk-segmented insurance, where premiums are set in accordance with health risks of the insured person, are being sold in the medical insurance and life insurance sectors. Periodically obtaining biometric information is desirable in risk-segmented insurance, in order to set appropriate premiums and to promote health of the insured person.

Devices in general households for measuring biometric information of the human body that are in widespread use include blood pressure meters, bathroom scales, body composition meters, activity monitors, and so forth. In order to use these measurement devices to compute insurance premiums, personal authentication needs to be performed in order to authenticate the person who has been measured to obtain the measured data (i.e., the measurement subject) is the insured person.

Technology that is usable as a way to provide personal authentication information with regard to measurement devices that do not have personal authentication functions, such as blood pressure meters, bathroom scales, and so forth, is being proposed (see International Publication No. 2014/115605). Technology where personal authentication information is succeeded, by comparing positional information of each of a measurement device having personal authentication functions and a measurement device to which attaching personal authentication information is desired, is disclosed in International Publication No. 2014/115605.

There also is a need for measurement values to accurately express biometric information of the measurement subject in order to use measurement values of devices in general households capable of measuring biometric information, such as blood pressure meters or the like, to compute insurance premiums. For example, with regard to technology to improve the accuracy of biometric information measurement at home, Japanese Patent Application Publication No. 2010-131305 discloses a blood pressure meter using an angle sensor, and Japanese Patent No. 5,249,273 discloses a measurement device equipped with a camera.

### SUMMARY

The following first problem is found in International Publication No. 2014/115605. That is to say, in a case where there are multiple users in a range where succession of personal authentication information is set to be enabled based on positional information, it is difficult for individual users to be identified by the technology in International Publication No. 2014/115605. Accordingly, a problem occurs where, in a case of a male and female living in the same house, and the male using the blood pressure meter and the female using the activity monitor at the same time, the measurement values of the blood pressure meter and the measurement values of the activity meter are personal-authenticated as being measurement values of the same person.

The following second problem is found in Japanese Patent Application Publication No. 2010-131305 and Japanese Patent No. 5,249,273. That is to say, in a case of measuring biometric information at home using household measurement devices, it is difficult to guarantee that the measured biometric information is accurate. On the other hand, traveling to a medical institution to measure biometric information places too great a burden on the measurement subject. Further, currently-available biometric information measurement devices assume that the user will use the devices to perform correct measurements, so it is difficult to prevent the user from intentionally performing incorrect measurements. For example, the technology in Japanese Patent Application Publication No. 2010-131305 that estimates the posture of the measurement subject using an angle sensor cannot tell the posture of the subject other than for the arm.

In one general aspect, the techniques disclosed here feature a blood pressure measurement state determination method for determining a blood pressure measurement state, using a determining device that determines a blood pressure measurement state and is held in a hand of a user to whom a blood pressure meter is mounted. The method includes: obtaining (i) image data including a face of the user by a camera that the device has, (ii) first information indicating an inclination angle of the determining device as to the gravitational direction, by an angle sensor that the device has, and (iii) second information indicating a position of the face of the user in the image data, and the proportion of the size of the face of the user in the image data; determining whether or not the user is correctly using the blood pressure meter based on the image data, the first information and the second information; and providing a notification indicating the determination result.

According to the present disclosure, personal authentication information can be succeeded from a first measurement device having personal authentication functions to a second measurement device not having personal authentication functions. Further, the posture of the user can be estimated more accurately as compared to posture estimation of the user using a conventional angle sensor, so the accuracy of measurement results is improved.

These general and specific aspects may be implemented using a system, a method, and a computer program, and any combination of systems, methods, and computer programs.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a block diagram schematically illustrating the configuration of a measurement subject identifying system according to a first embodiment;

FIG. 2 is a diagram schematically illustrating an example of measurement data stored in a measurement data database;

FIG. 3 is a sequence diagram schematically illustrating an example of authentication procedures by ID and password;

FIG. 4 is a diagram schematically illustrating exchange of signals between a server and measurement device and a display example on a measurement device;

FIG. 5 is a sequence diagram schematically illustrating an example of authentication procedures by fingerprint;

FIG. 6 is a sequence diagram schematically illustrating an example of authentication procedures by facial image;

FIG. 7 is a sequence diagram schematically illustrating an example of succession procedures for personal authentication information;

FIG. 8 is a flowchart schematically illustrating an example of operation procedures of a similarity verifying unit of a server;

FIG. 9 is a diagram schematically illustrating an example of measurement data stored in a measurement value database that differs from the example in FIG. 2;

FIG. 10 is a flowchart schematically illustrating an example of procedures for detecting pulse from a moving image.

FIG. 11 is a diagram illustrating an example of updating a measurement value database in a case where personal authentication information has been succeeded;

FIG. 12 is a sequence diagram of procedures notifying use as a personal-authenticated device;

FIG. 13 is a diagram schematically illustrating exchange of signals between a server and measurement device and a display example on a measurement device;

FIG. 14 is a flowchart schematically illustrating another example of procedures for attaching personal authentication information;

FIG. 15 is a sequence diagram schematically illustrating operations when a measurement device that has succeeded personal authentication information has ended measurement of biometric information;

FIG. 16 is a diagram schematically illustrating an example of authentication management information;

FIG. 17 is a sequence diagram schematically illustrating an example of personal authentication information succession procedures according to a second embodiment;

FIG. 18 is a diagram illustrating a specific updating example of a measurement value database in the second embodiment;

FIG. 19 is a sequence diagram schematically illustrating an example of personal authentication information attaching procedures according to a third embodiment;

FIG. 20 is a block diagram schematically illustrating an example of the configuration of a determining device according to a fourth embodiment;

FIG. 21 is a block diagram schematically illustrating an example of the configuration of a blood pressure meter;

FIG. 22 is a diagram schematically illustrating a measurement state when a user uses the blood pressure meter;

FIG. 23 is a diagram schematically illustrating an example of a screen displayed on a display unit before starting measurement;

FIG. 24 is a flowchart schematically illustrating operations of the determining device according to the fourth embodiment;

FIG. 25 is a diagram schematically illustrating an example of a screen displayed on the display unit when measuring blood pressure;

FIG. 26 is a diagram schematically illustrating an example of a screen displayed on the display unit when measuring blood pressure;

FIG. 27 is a diagram schematically illustrating an example of a screen displayed on the display unit when measuring blood pressure;

FIG. 28 is a diagram schematically illustrating an example of a screen displayed on the display unit when measuring blood pressure;

FIG. 29 is a block diagram schematically illustrating an example of the configuration of a determining device according to a fifth embodiment;

FIG. 30 is a diagram schematically illustrating a measurement state when a user uses the blood pressure meter;

FIG. 31 is a diagram schematically illustrating an example of a screen displayed on a display unit before starting measurement;

FIG. 32 is a flowchart schematically illustrating operations of the determining device according to the fifth embodiment;

FIG. 33 is a diagram schematically illustrating an example of a screen displayed on the display unit when measuring blood pressure;

FIG. 34 is a diagram schematically illustrating an example of a screen displayed on the display unit when measuring blood pressure;

FIG. 35 is a diagram schematically illustrating an example of a screen displayed on the display unit when measuring blood pressure;

FIG. 36 is a block diagram schematically illustrating an example of the configuration of a determining device according to a sixth embodiment;

FIG. 37 is a flowchart schematically illustrating operations of a determining device according to the sixth embodiment; and

FIG. 38 is a block diagram illustrating an example of the configuration of a system having a determining device and a server.

### DETAILED DESCRIPTION

### Underlying Knowledge Forming Basis of the Present Disclosure

There is being studied technology in the life insurance and medical insurance sector where the health state of an insured person is accurately analyzed and comprehended by measuring biometric information of the insured person by measurement devices, and thereby computing insurance premiums appropriated for the insured person. There are measurement devices capable of measuring biometric information of an insured person that do not have functions to guarantee that the measured biometric information is that of the insured person themselves.

For example, a type of medical insurance will be considered where an insurance provider distributes activity monitors to insured persons, measures the number of steps walked by the insured person along with the pulse, and reduces the insurance premium in a case where the number of steps walked exceeds 10,000 every day. A situation can be conceived in this medical insurance where the insured person gives the activity monitor distributed from the insurance provider to a person other than the insured person. In this case, if the insured person does not walk 10,000 steps every day but the person who was given the activity monitor does walk 10,000 steps every day, a situation will occur where the insurance premium of the insured person is erroneously reduced.

As another example, a type of medical insurance will be considered where an insurance provider distributes blood pressure meters to insured persons, and in a case an insured person whose systolic blood pressure was 140 mmHg or higher, which is the borderline for diagnosis of high blood pressure, was able to be reduced to 129 mmHg or lower, which is in a normal blood pressure range through healthy living, while being insured, the insurance premium is reduced. It is important for this health insurance that the blood pressure value to be correctly measured without falsification. However, many existing blood pressure meters will measure the blood pressure value lower than it actually is, by the user raising the wrist or upper arm wearing the compression sleeve (hereinafter also referred to as “cuff”) where the blood pressure is measured above the height of the heart when measuring. Accordingly, there is a possibility that a situation will occur where insurance premiums are reduced for insured persons who should not receive reduction in insurance premiums.

In the above examples, no function exists in the activity monitors or blood pressure meters distributed by the insurance providers to identify that the measurement subject is the insured person. Accordingly, a situation can occur where measurement values by measurement subjects other than the insured person are erroneously used for computation of insurance premiums. Thus, there is a problem in that the biometric information has to be identified as having been measured by the insured person, in order to apply the measured biometric information to computation of insurance premiums.

International Publication No. 2014/115605 proposes, as an individual authentication technology capable of solving such problems, propagating authentication information in a personal-authenticated measurement device to a measurement device where personal authentication is not performed, by comparing positional information of each measurement device among multiple measurement devices, and handling as personal-authenticated measuring device. It is difficult to determine whether or not positional information among the multiple measurement devices precisely matches, as stated in International Publication No. 2014/115605. Accordingly, a configuration is made in International Publication No. 2014/115605 where personal authentication information can be propagated when the locational positions of the respective measurement devices are within a predetermined range.

In a case where succession of personal authentication information is to be performed among multiple measurement devices that measure biometric information, based on positional information between a blood pressure meter and activity monitor for example, succession of personal authentication information is performed in a case where the blood pressure meter and the activity monitor exist within a range that has been set to enable succession of personal authentication information. However, simply having the blood pressure meter and activity monitor within the range that has been set to enable succession of personal authentication information cannot guarantee that the subject of measurement of these two measurement devices is the same person. Thus, International Publication No. 2014/115605 has not sufficiently studied a way to guarantee that that the subject of measurement is the same person for multiple measurement devices regarding which succession of personal authentication information is desired, situated within the range that has been set to enable succession of personal authentication information.

In order to solve this problem, the Present Inventor has studied the following improvements.

(1) A first aspect of the present disclosure is a measurement subject identifying method for identifying a measurement subject at a measurement device that is connected to a network and that measures vital data of the measurement subject,

wherein first vital data measured at a first measurement device capable of personal authentication of a user, and personal authentication information of the user personal-authenticated by the first measurement device, are received from the first measurement device via the network, the first vital data including measurement data of at least one measurement type,

wherein second vital data measured at a second measurement device that is different from the first measurement device is received from the second measurement device via the network, the second vital data including measurement data of at least the measurement type included in the first vital data,

wherein a first value indicating the degree of matching of each measurement data of the mutually common measurement type included in the first vital data and the second vital data is calculated,

wherein determination is made regarding whether the first value is greater than a first threshold value set beforehand,

and wherein, in a case where the first value is greater than the first threshold value, the second vital data is identified as being measurement data of the user of the first measurement device personal-authenticated by the first measurement device, and is stored in memory with the second vital data and the personal authentication information correlated.

According to this aspect, personal authentication information can be succeeded from the first measurement device capable of personal authentication to the second measurement device that does not have personal authentication functions, based on the first vital data and the second vital data of the user personal-authenticated by the first measurement device.

(2) In the above-described first aspect, for example, the at least one measurement type may include at least one of blood pressure, number of steps walked, pulse, heart rate, electrocardiogram, respiratory rate, weight, and depth of sleep.

(3) In the above-described first aspect, the received personal authentication information may include, for example, at least one of user identification information and password, fingerprint of user, and facial image of user.

According to this aspect, succeeded personal information can be made to be correct.

(4) In the above-described first aspect, for example, a command to display information indicating that the measurement subject using the second measurement device has been identified as a user of the first measurement device, may be transmitted to the second measurement device via the network.

According to this aspect, when information indicating that the measurement subject using the second measurement device has been identified as a user of the first measurement device is displayed, the measurement subject using the second measurement device can know that personal authentication has been succeeded.

(5) In the above-described first aspect, for example, third vital data measured at a third measurement device that differs from the first measurement device and the second measurement device may further be received from the third measurement device via the network. The third vital data may include measurement data of a measurement type included in at least the second vital data. A second value indicating the degree of matching of each measurement data of the mutually common measurement type included in the second vital data and the third vital data may be calculated. Determination may be made regarding whether the second value is greater than a second threshold value set beforehand. In a case where the second value is greater than the second threshold value, the third vital data may be identified as being measurement data of the user of the first measurement device personal-authenticated by the first measurement device, and may be stored in memory with the third vital data and the personal authentication information correlated.

According to this aspect, personal authentication information of the user personal-authenticated by the first measurement device can be further succeeded by the third measurement device, by comparing measurement data of a measurement type measured in common between the second measurement device and third measurement device.

(6) A second aspect of the present disclosure is a measurement subject identifying system that identifies a measurement subject at a measurement device that is connected to a network and measures vital data of the measurement subject, including

a first measurement device that is connected to the network and that measures a first vital data including at least one measurement type of measurement data of the measurement subject,

a second measurement device that is different from the first measurement device, that is connected to the network, and that measures a second vital data including at least measurement data of a measurement type included in the first vital data of the measurement subject, and

a management device connected to the network,

wherein the management device includes
a reception unit that receives, via the network, the first vital data measured at the first measurement device from the first measurement device, personal identification information of the user personal-authenticated by the first measurement device, and the second vital data measured at the second measurement device from the second measurement device via the network,
a processing unit, and
memory,

and wherein the processing unit includes
a calculating unit that calculates a first value indicating the degree of matching of each measurement data of a mutually common measurement type included in the first vital data and the second vital data,
a determining unit that determines whether the first value is greater than a first threshold value set beforehand and stored in the memory,
a management unit that, in a case where the first value is greater than the first threshold value, identifies the second vital data as being measurement data of the user of the first measurement device personal-authenticated by the first measurement device, and stores in the memory with the second vital data and the personal authentication information correlated.

(7) In the above-described second aspect, for example, the at least one measurement type may include at least one of blood pressure, number of steps walked, pulse, heart rate, electrocardiogram, respiratory rate, weight, and depth of sleep.

Also, the medical insurance using the blood pressure meter exemplified in the Present Underlying Knowledge, a way to confirm that blood pressure is being measured in a state where the compression sleeve of the blood pressure meter is situated at the height of the heart is necessary. The technology of Japanese Patent Application Publication No. 2010-131305 estimates the height of the position where the cuff is mounted, by attaching an angle sensor to the cuff of a wrist-measurement blood pressure meter. Assuming that the upper arm is fixed in a natural posture, the height of the cuff is decided by the angle of the forearm, so the height of the cuff can be estimated by detecting the angle of the forearm by the angle sensor. The blood pressure measurement results are dependent on the height of the cuff, so the height of the cuff can be made to be constant and thereby improve reliability of the results of blood pressure measurement.

Also, in the technology described in Japanese Patent No. 5,249,273, personal authentication of the user can be performed by facial recognition, by mounting a camera on the measurement device of biometric information, of which a blood pressure meter is representative. According to this method, measurement results and measurement device settings can be associated with the person, thereby facilitating management of measurement data.

However, the technologies in Japanese Patent Application Publication No. 2010-131305 and Japanese Patent No. 5,249,273 assume that the user will use the measurement devices to measure correct values. Accordingly, it has been easy to intentionally perform measurement by incorrect measurement methods, and obtain fraudulent measurement values. In the technology described in the above Japanese Patent Application Publication No. 2010-131305, changing the position of the forearm enables the actual height of the cuff worn on the wrist to be changed, without changing the height of the cuff estimated by the blood pressure meter. As a result, the measurement values of blood pressure can be intentionally raised or lowered. Also, in the technology described in Japanese Patent No. 5,249,273, the position and orientation of the camera cannot be fixed, so posture estimation cannot be made other than when sitting, such as regarding lying down or the like. As a result, it is easy to intentionally perform incorrect measurements.

Accordingly, the Present Inventor has studied the following improvements.

(8) A third aspect of the present disclosure is a blood pressure measurement state determination method for determining a blood pressure measurement state, using a determining device that determines a blood pressure measurement state and is held in a hand of a user to whom a blood pressure meter is mounted, the method including

obtaining image data including a face of the user by a camera that the determining device has,

obtaining first information indicating an inclination angle of the determining device as to the gravitational direction, by an angle sensor that the determining device has,

obtaining second information indicating a position of the face of the user in the image data, and the proportion of the size of the face of the user in the image data,

determining whether or not the angle indicated in the first information is within a first range,

determining whether or not the position of the face of the user indicated in the second information is within a second range,

determining whether or not the proportion of the size of the face of the user indicated in the second information is within a third range,

determining whether or not the user is correctly using the blood pressure meter,

notifying the user that the blood pressure meter is being correctly used upon determination being made that the user is correctly using the blood pressure meter, and

prompting the user to use the blood pressure meter correctly upon determination being made that the user is not correctly using the blood pressure meter,

wherein in the determination regarding whether or not the user is correctly using the blood pressure meter, determination is made that the user is correctly using the blood pressure meter when the angle indicated in the first information is determined to be within the first range, the position of the face of the user indicated in the second information is determined to be within the second range, and the proportion of the size of the face of the user indicated in the second information is determined to be within the third range.

According to this third aspect, using the image of the camera that the determining device held in the hand of the user has enables the posture of the user to be estimated more accurately as compared to posture estimation of the user by a conventional angle sensor alone, and the precision of measurement results is improved.

Also, an image of the face of the user is used, so difference in postures that the angle sensor alone cannot estimate can be detected, making it more difficult for the user to intentionally make fraudulent measurements. As a result, fraudulence that is readily conceivable, such as raising the arm or measuring while lying down or the like can be eliminated, thereby enabling fraudulent measurements to be suppressed.

Further, correct usage of the blood pressure meter can be quantitatively expressed by angle information of the determining device, the position of the face in image data, and the proportion of the face in the image data. Consequently, measures to take for the user to correctly use can be easily instructed.

(9) In the above-described third aspect, for example, audio around the determining device may further be obtained. Determination may be performed regarding whether or not the obtained audio and audio emitted by the blood pressure meter, stored in a storage unit beforehand, agree. In the determining of whether or not the user is correctly using the blood pressure meter correctly, determination may further be made that the user is using the blood pressure meter correctly if determination is made that the obtained audio and audio emitted by the blood pressure meter stored in the storage unit agree.

According to this aspect, comparing the obtained audio with the audio emitted by the blood pressure meter enables confirmation to be made whether measurement is being performed by the blood pressure meter near the determining device. As a result, the reliability of determination regarding whether or not the blood pressure meter is being correctly used improves. For example, in a case where wearing the blood pressure meter on the same arm as the hand holding the determining device is recommended, fraudulent measurement such as mounting the blood pressure meter to the other arm as the hand holding the determining device can be prevented.

(10) In the above-described third aspect, for example, further, a command instructing to start measurement of blood pressure of the user may be transmitted to the blood pressure meter upon determination being made that the user is using the blood pressure meter correctly.

According to this aspect, blood pressure measurement can be started without the user having to move, so it is easier for the user to maintain a correct posture. As a result, precision of measurement can be improved, and the load on the user can be reduced.

(11) In the above-described third aspect, for example, further, determination may be made again regarding whether or not the user is using the blood pressure meter correctly, after a predetermined amount of time has elapsed after transmitting the command.

According to this aspect, in a case where the user has ceased to correctly use the blood pressure meter after starting measurement, this is detected, whereby determination precision of the blood pressure measurement state can be improved.

(12) In the above-described third aspect, for example, further, determination may be made again regarding whether or not the user is using the blood pressure meter correctly, upon receiving a notification is received from the blood pressure meter indicating that blood pressure measurement of the user has ended.

According to this aspect, in a case where the user has ceased to correctly use the blood pressure meter at the time of ending the blood pressure measurement, this is detected, whereby determination precision of the blood pressure measurement state can be improved.

(13) In the above-described third aspect, for example, further, a first icon indicating the position to hold the determining device with one hand out of the left hand and right hand of the user, and a second icon indicating the position to hold the determining device with other hand out of the left hand and right hand of the user, may be displayed on a touch panel display unit provided to the determining device. In the determination regarding whether or not the user is using the blood pressure meter correctly, further, determination may be made that the user is using the blood pressure meter correctly if detecting that the one hand of the user is in contact with the first icon and that the other hand of the user is in contact with the second icon.

According to this aspect, the postures that the user can assume can be restricted by causing the user to hold the determining device at predetermined position with both hands. As a result, the user can be naturally led to correctly use the blood pressure meter in the correct posture.

(14) In the above-described third aspect, for example, further, determination may be made regarding whether or not the image data matches image data of the user of the determining device stored in the storage unit beforehand. In the determination regarding whether or not the user is using the blood pressure meter correctly, further, determination may be made that the user is using the blood pressure meter correctly if detecting that the image data matches image data of the user of the determining device stored in the storage unit.

According to this aspect, performing personal authentication of the user enables the measurement value of blood pressure to be associated with an individual, and can be used for purposes requiring measurement values of particular individuals, such as computation of insurance premiums or the like.

(15) In the above-described third aspect, for example, further, a notification indicating that the user is correctly using the blood pressure meter may be transmitted to a server connected to the determining device upon determination being made that the user is using the blood pressure meter correctly.

According to this aspect, appropriate diagnosis and insurance premium calculation in accordance with the reliability of measurement values is enabled by externally transmitting reliability of blood pressure values to medical institutions, insurance providers, and so forth, such as ignoring measurement values with low reliability or the like.

(16) A fourth aspect of the present disclosure is a blood pressure measurement state determining device for determining a blood pressure measurement state that is held in a hand of a user to whom a blood pressure meter is mounted, the determining device including

a camera that obtains image data including a face of the user,

an angle sensor that obtains first information indicating an inclination angle of the determining device as to the gravitational direction,

a processing unit, and

and a display unit,

wherein the processing unit includes
a facial position determining unit that obtains second information indicating a position of the face of the user in the image data, and the proportion of the size of the face of the user in the image data,
a posture determining unit that determines whether or not the angle indicated in the first information is within a first range, determines whether or not the position of the face of the user indicated in the second information is within a second range, and determines whether or not the proportion of the size of the face of the user indicated in the second information is within a third range, and
a reliability determining unit that determines that the user is correctly using the blood pressure meter if determination is made that the angle indicated in the first information is within a first range, determination is made that the position of the face of the user indicated in the second information is within a second range, and determination is made that the proportion of the size of the face of the user indicated in the second information is within a third range,

and wherein the display unit
displays a notification to the user indicating that the blood pressure meter is being correctly used upon determination being made that the user is correctly using the blood pressure meter, and
displays a notification prompting the user to use the blood pressure meter correctly upon determination being made that the user is not correctly using the blood pressure meter.

(17) A fifth aspect of the present disclosure is a blood pressure measurement state determination program for determining a blood pressure measurement state, using a determining device that determines a blood pressure measurement state and is held in a hand of a user to whom a blood pressure meter is mounted,

the blood pressure measurement state determination program causing a computer of the determining device to execute
obtaining image data including a face of the user by a camera that the determining device has,
obtaining first information indicating an inclination angle of the determining device as to the gravitational direction, by an angle sensor that the determining device has,
obtaining second information indicating a position of the face of the user in the image data, and the proportion of the size of the face of the user in the image data,
determining whether or not the angle indicated in the first information is within a first range,
determining whether or not the position of the face of the user indicated in the second information is within a second range,
determining whether or not the proportion of the size of the face of the user indicated in the second information is within a third range,
determining whether or not the user is correctly using the blood pressure meter,
notifying the user that the blood pressure meter is being correctly used upon determination being made that the user is correctly using the blood pressure meter, and
prompting the user to use the blood pressure meter being upon determination being made that the user is not correctly using the blood pressure meter,

wherein in the determination regarding whether or not the user is correctly using the blood pressure meter, determination is made that the user is not correctly using the blood pressure meter when the angle indicated in the first information is determined to be within the first range, the position of the face of the user indicated in the second information is determined to be within the second range, and the proportion of the size of the face of the user indicated in the second information is determined to be within the third range.

These general or specific aspects may be implemented as a method where feature configurations included in the above-described device are carried out as steps, or as a computer program where feature configurations included in the above-described device are executed by a computer. These may also be realized by a computer-readable non-transitory recording medium such as a CD-ROM in which such a computer program is recorded, may be distributed via a communication network such as the Internet or the like, or may be constructed as a cloud computing system where multiple computers operate in a decentralized manner. Further, these may be realized by any combination of a system, method, integrated circuit, computer program, and recording medium.

Embodiments for carrying out the present disclosure will be described below with reference to the drawings. In all of the following drawings, same reference symbols denote the same object, unless specifically indicated otherwise. Note that the embodiments described below are all specific examples of the present disclosure. Values, shapes, components, steps, the order of steps, and so forth, illustrated in the following embodiments, are only exemplary, and do not restrict the present disclosure. Components in the following embodiments which are not included in an independent Claim are described as being optional components. The contents of each and all of the embodiments may be combined.

### First Embodiment

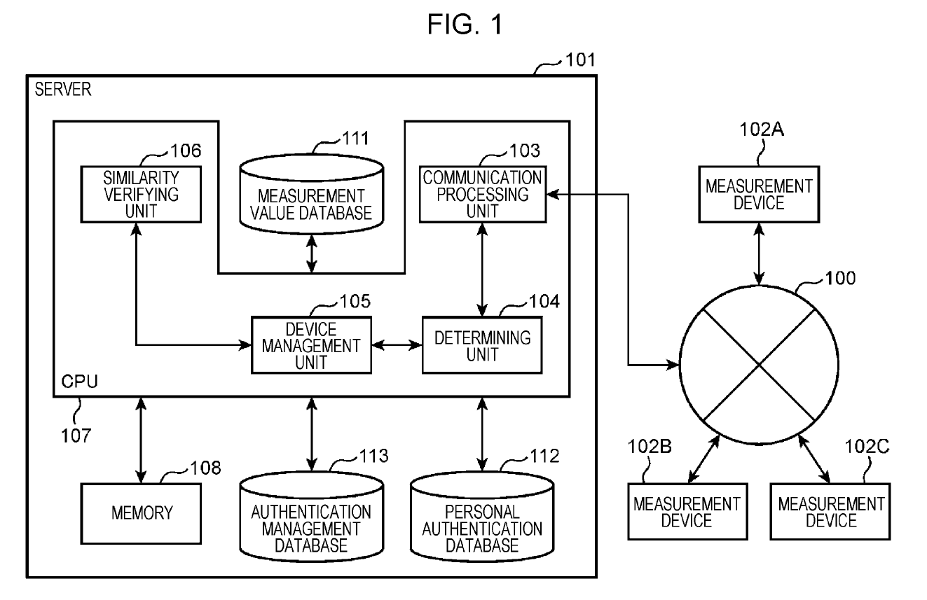

FIG. 1 is a block diagram schematically illustrating the configuration of a measurement subject identifying system according to a first embodiment. The measurement subject identifying system illustrated in FIG. 1 includes a server 101 and multiple measurement devices 102A, 102B, and 102C. The server 101 and multiple measurement devices 102A, 102B, and 102C are each connected via a network 100. The server 101 causes personal authentication information to be succeeded among the multiple measurement devices 102A, 102B, and 102C. The multiple measurement devices 102A, 102B, and 102C each measure vital data of a measurement subject. Specific examples of vital data will be described later.

The server 101 (equivalent to an example of a management device) includes a central processing unit (CPU) 107, memory 108, a measurement value database 111, a personal authentication database 112, and an authentication management database 113, as illustrated in FIG. 1. The server 101 may be configured of a personal computer.

The memory 108 is made up of semiconductor memory or the like, for example. The memory 108 includes, for example, read-only memory (ROM), random access memory (RAM), electrically erasable programmable read-only memory (EEPROM), and so forth. The ROM of the memory 108 stores a control program according to the present embodiment to operate the CPU 107.

The CPU 107 (equivalent to an example of a processing unit) has the functions of a communication processing unit 103, a determining unit 104, a device management unit 105, and a similarity verifying unit 106, by operating following the control program of the present embodiment, stored in the memory 108. The functions of the parts of the CPU 107 will be described later. Note that the server 101 may have other hardware that realizes the same functions, instead of the CPU 107.

The measurement value database 111, personal authentication database 112, and authentication management database 113 are configured of a hard disk or semiconductor memory or the like. Data stored in the measurement value database 111, personal authentication database 112, and authentication management database 113 will be described later. The measurement value database 111, personal authentication database 112, and authentication management database 113 may each be configured of mutually different media. Alternatively, the measurement value database 111, personal authentication database 112, and authentication management database 113 may be configured of a single medium of which the storage region has been partitioned.

The communication processing unit 103 (equivalent to an example of a reception unit) has communication functions of communicating with the measurement devices 102A, 102B, and 102C via the network 100. The communication processing unit 103 receives measurement data transmitted from the measurement devices 102A, 102B, and 102C. The communication processing unit 103 stores the received measurement data in the measurement value database 111.

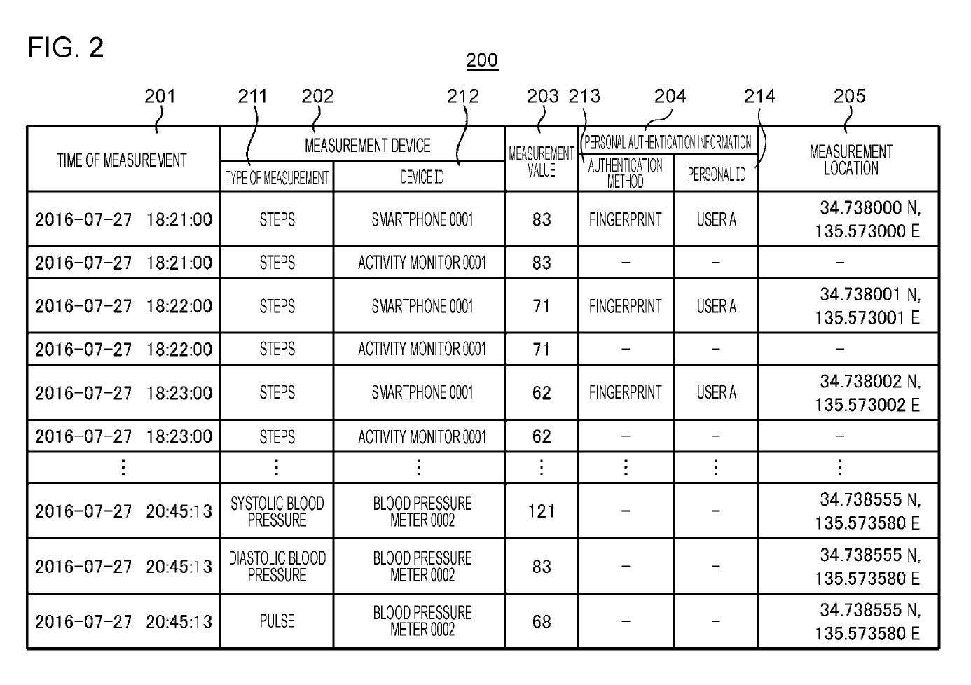

FIG. 2 is a diagram schematically illustrating an example of measurement data 200 stored in the measurement value database 111 (equivalent to an example of memory). The measurement data 200 includes a time-of-measurement space 201, a measurement device space 202, a measurement value space 203, a personal authentication information space 204, and a measurement location space 205. The measurement device space 202 has a type-of-measurement space 211 and a device ID space 212. The personal authentication information space 204 includes an authentication method space 213, and a personal ID space 214.

The time-of-measurement space 201 records time of measurement. The type-of-measurement space 211 of the measurement device space 202 stores types of vital data. The device ID space 212 of the measurement device space 202 stores authentication information (IDs) uniquely identifying the measurement devices 102A, 102B, and 102C. The measurement values in the vital data are recorded in the measurement value space 203. The authentication method space 213 of the personal authentication information space 204 records the authentication method used for personal authentication of the measurement device, in a case where the corresponding measurement device is configured to be capable of personal authentication. The personal ID space 214 of the personal authentication information space 204 records authentication information (IDs) uniquely identifying the users of the measurement devices. In a case where the corresponding device has functions of obtaining position information, such as having global positioning system (GPS) reception functions for example, the measurement location space 205 records the latitude and longitude indicating the position of the measurement device.

The determining unit 104 receives information necessary for personal authentication from the measurement devices 102A, 102B, and 102C via the communication processing unit 103. The determining unit 104 compares received information with information saved in the personal authentication database 112, and determines whether or not the measurement devices 102A, 102B, and 102C, which are the recipients of the information, have been personal-authenticated. The determining unit 104 notifies the device management unit 105 of the results of determination, and transmits to the measurement devices 102A, 102B, and 102C via the communication processing unit 103.

The similarity verifying unit 106 (equivalent to an example of a calculating unit and determining unit) reads measurement data 200 stored in the measurement value database 111, and extracts features from measurement values for each ID in the device ID space 212 over a predetermined period. The similarity verifying unit 106 calculates the similarity of features (equivalent to an example of a first value indicating degree of matching) for each extracted ID. When the similarity calculated between a personal-authenticated device ID and an unauthenticated device ID exceeds a predetermined first threshold value, the similarity verifying unit 106 determines the unauthenticated device ID to have been personal-authenticated, and notifies the device management unit 105 of the personal-authenticated device ID.

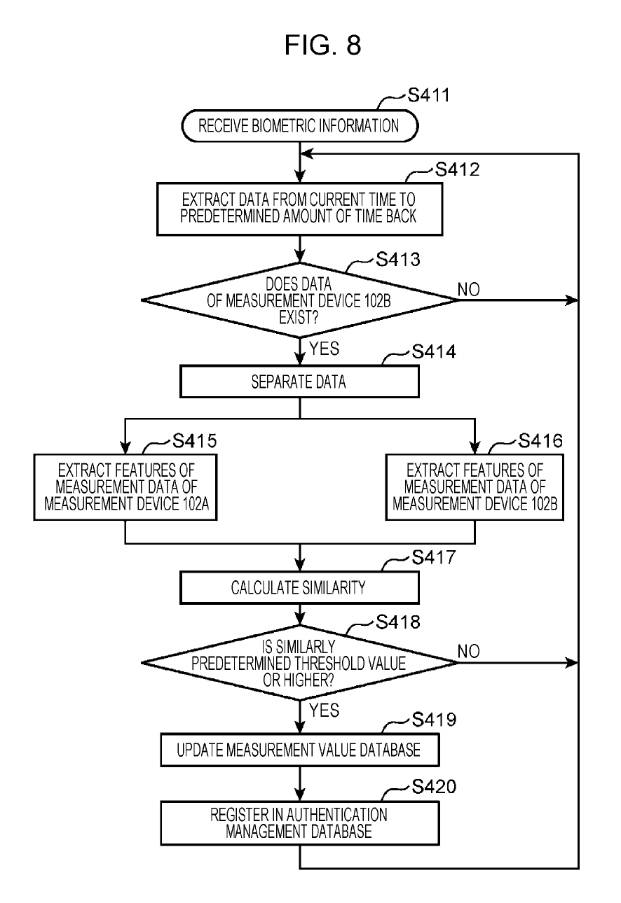

The device management unit 105 (equivalent to an example of a management unit) receives information regarding whether or not the measurement devices 102A, 102B, and 102C are personal-authenticated, from the determining unit 104 and similarity verifying unit 106. Based on the received information, the device management unit 105 updates the personal authentication information space 204 in the measurement value database 111. Updating of personal authentication information in the measurement value database 111 by the device management unit 105 will be specifically exemplified by description in FIG. 8.

The personal authentication database 112 stores data regarding which an individual can be identified, by the determining unit 104 comparing information necessary for personal authentication received from the measurement device 102A via the communication processing unit 103, and data stored in the personal authentication database 112. Examples of personal authentication formats that the personal authentication database 112 can use include personal authentication identification information (ID) and password, biometric personal authentication by fingerprint information, biometric personal authentication by facial image, and so forth.

In the personal authentication by ID and password, the personal authentication database 112 stores information for matching IDs and passwords. In the biometric personal authentication by fingerprint information, the personal authentication database 112 stores features information by which individuals can be uniquely identified from fingerprint information. In the biometric personal authentication by facial image, the personal authentication database 112 stores features information by which individuals can be uniquely identified from facial images. The personal authentication database 112 may have one or multiple personal authentication formats as the personal authentication format. Note that the personal authentication formats that the personal authentication database 112 has is not restricted to the exemplified personal authentication formats.

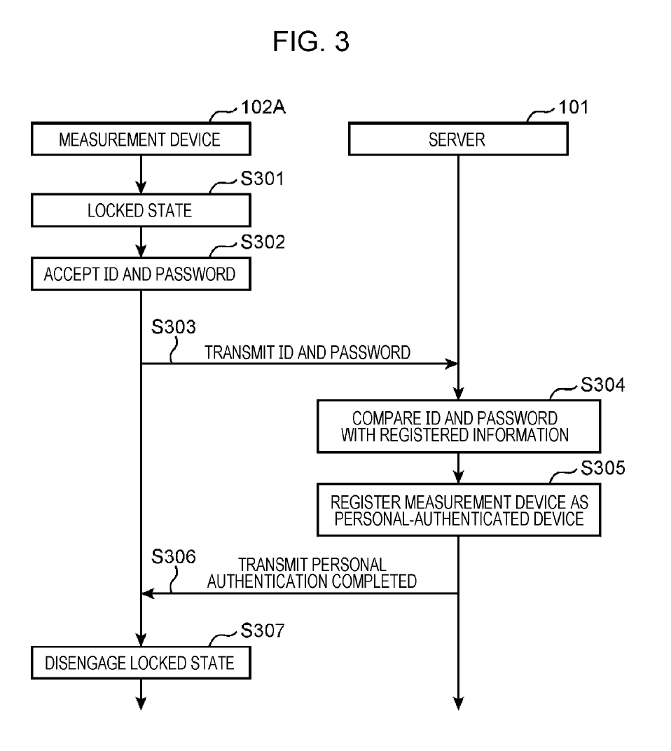

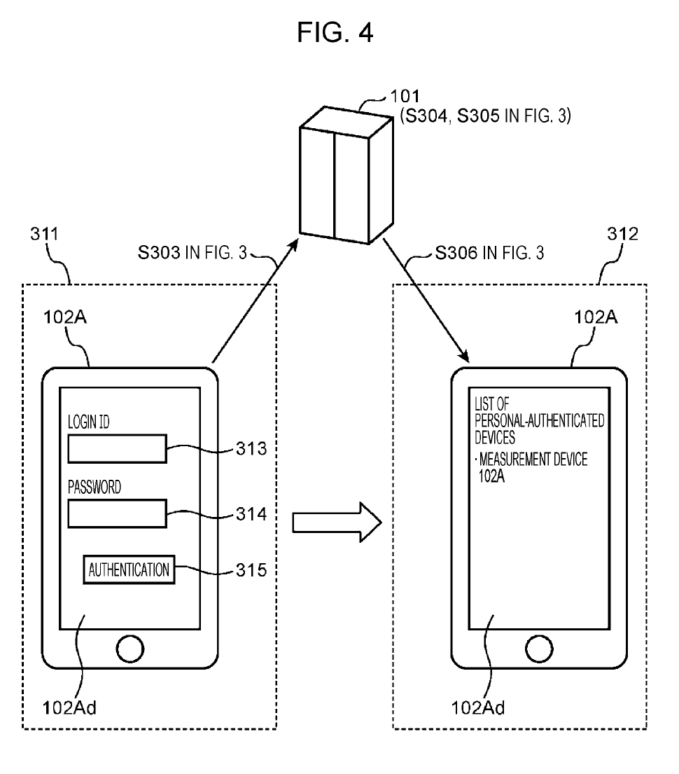

FIG. 3 is a sequence diagram schematically illustrating an example of authentication procedures in the personal authentication by ID and password in the determining unit 104. The measurement device 102A is in a state where personal authentication has not been performed and the device is locked in step S301. In the locked state in step S301, the measurement device 102A accepts an ID and password input by the user in step S302. In step S303, the ID and password are transmitted from the measurement device 102A to the server 101. The determining unit 104 of the server 101 compares the received ID and password with the personal authentication information registered in the personal authentication database 112 in step S304, and determines whether or not these match. The determining unit 104 notifies the results of determination to the device management unit 105.

In a case where the received ID and password match the personal authentication information, step S305 is executed, and the device management unit 105 of the server 101 registers the measurement device 102A in the measurement value database 111 and authentication management database 113 as a personal-authenticated device. In a case where step S305 is executed in step S306, the device management unit 105 of the server 101 transmits a signal representing completion of personal authentication to the measurement device 102A via the communication processing unit 103. In step S307, the locked state of the measurement device 102A is disengaged.

On the other hand, in a case where the received ID and password do not match the personal authentication information in step S304, step S305 is not executed, and the device management unit 105 of the server 101 transmits a signal indicating that the personal authentication information does not match, to the measurement device 102A via the communication processing unit 103. The measurement device 102A transitions to the locked state of step S301, and goes to a state of prompting the user for input of ID and password again.

FIG. 4 is a diagram schematically illustrating an example of exchange of signals between the server 101 and measurement device 102A when the processing illustrated in the sequence diagram in FIG. 3 is being executed, and a display example on the measurement device 102A. In the unauthenticated locked state 311, the user inputs a login ID to an ID input space 313 of a touch-panel display unit 102Ad of the measurement device 102A for example, inputs a password to a password input space 314, and taps an authentication button 315, whereby step S303 is executed, and the ID and password are transmitted from the measurement device 102A to the server 101. In a case where steps S304 and S305 are executed at the server 101, a signal indicating completion of personal authentication is transmitted from the server 101 to the measurement device 102A in step S306. Upon the measurement device 102A receiving the personal authentication completion, the measurement device 102A transitions to a lock-disengaged state 312, a list of personal-authenticated devices is displayed on the display unit 102Ad of the measurement device 102A, where the measurement device 102A is displayed.

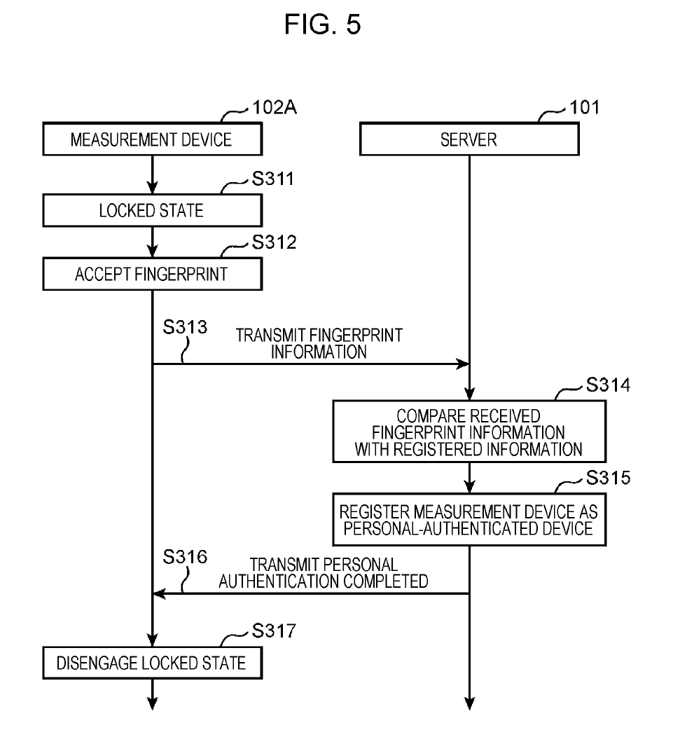

FIG. 5 is a sequence diagram schematically illustrating an example of authentication procedures in biometric personal authentication by fingerprint in the determining unit 104. In a case of performing biometric personal authentication by fingerprint, the measurement device 102A needs to be provided with fingerprint reading functions. In a case where the measurement device 102A does not have fingerprint reading functions, the measurement device 102A cannot perform biometric personal authentication by fingerprint, but having fingerprint reading functions is not an indispensable condition for the measurement device 102A.

In step S311, the measurement device 102A is in a state where personal authentication has not been performed and the device is locked. In the locked state in step S311, the measurement device 102A accepts a fingerprint input by the user in step S312. The fingerprint information input by the user is transmitted from the measurement device 102A to the server 101 in step S313. In step S314, the determining unit 104 of the server 101 compares features of the received fingerprint information with personal authentication information registered in the personal authentication database 112, and determines whether these match or not. The determining unit 104 notifies the device management unit 105 of the results of determination.

In a case where the features of the received fingerprint information match the personal authentication information, step S315 is executed, and the device management unit 105 of the server 101 registers the measurement device 102A in the measurement value database 111 and authentication management database 113 as a personal-authenticated device. In a case where step S315 is executed, the device management unit 105 of the server 101 transmits a signal indicating completion of personal authentication to the measurement device 102A via the communication processing unit 103 in step S316. In step S317, the locked state of the measurement device 102A is disengaged.

On the other hand, in a case where the features of the received fingerprint information do not match the personal authentication information in step S314, step S315 is not executed, and the device management unit 105 of the server 101 transmits a signal to the measurement device 102A via the communication processing unit 103, indicating that the personal authentication information does not match. The measurement device 102A transitions to the locked state in step S311, and goes to a state of prompting the user for input of a fingerprint again.

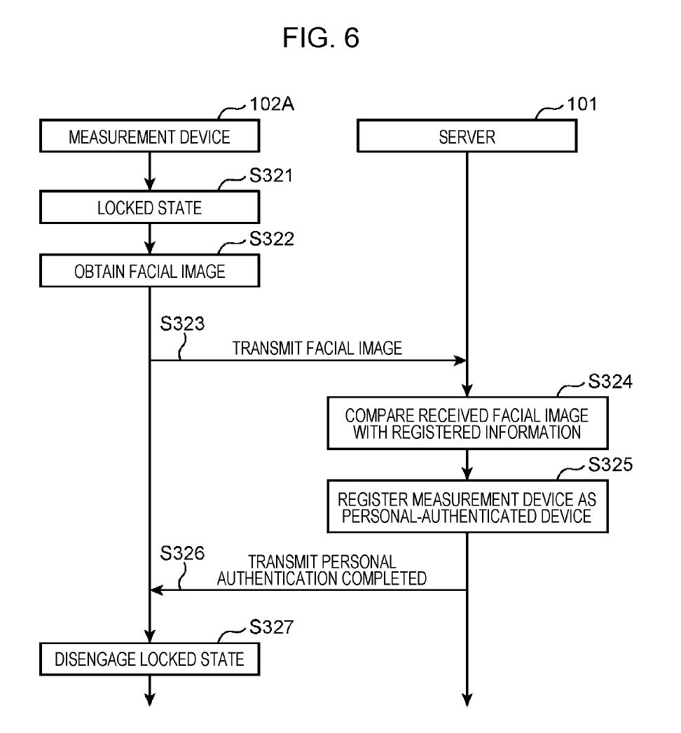

FIG. 6 is a sequence diagram schematically illustrating an example of authentication procedures in biometric personal authentication by facial image in the determining unit 104. In a case of performing biometric personal authentication by facial image, the measurement device 102A needs to be provided with a camera. In a case where the measurement device 102A does not have a camera, the measurement device 102A cannot perform biometric personal authentication by facial image, but having a camera is not an indispensable condition for the measurement device 102A.

In step S321, the measurement device 102A is in a state where personal authentication has not been performed and the device is locked. In the locked state in step S321, the measurement device 102A prompts the user to take a facial image of the user, using the camera that the measurement device 102A has, in step S322. The measurement device 102A obtains a facial image of the user that has been taken. The facial image of the user that has been taken is transmitted from the measurement device 102A to the server 101 in step S323. In step S324, the determining unit 104 of the server 101 compares features of the received facial image with personal authentication information registered in the personal authentication database 112, and determines whether these match or not. The determining unit 104 notifies the device management unit 105 of the results of determination.

In a case where the features of the received facial image match the personal authentication information, step S325 is executed, and the device management unit 105 of the server 101 registers the measurement device 102A in the measurement value database 111 and authentication management database 113 as a personal-authenticated device. In a case where step S325 is executed, the device management unit 105 of the server 101 transmits a signal indicating completion of personal authentication to the measurement device 102A via the communication processing unit 103 in step S326. In step S327, the locked state of the measurement device 102A is disengaged.

On the other hand, in a case where the features of the received facial image do not match the personal authentication information in step S324, step S325 is not executed, and the device management unit 105 of the server 101 transmits a signal to the measurement device 102A via the communication processing unit 103, indicating that the personal authentication information does not match. The measurement device 102A transitions to the locked state in step S321, and goes to a state of prompting the user to take a facial image again.

The measurement device 102A can transmit measurement data representing biometric information (vital data) to the server 101 as a personal-authenticated device, by the techniques exemplified in FIGS. 3, 5, and 6. While the measurement device 102A is being recognized by the server 101 as a personal-authenticated device, the data that the server 101 has received from the measurement device 102A is stored in the server 101 with the personal authentication information space 204 filled in, following the format exemplified in FIG. 2. That is to say, as illustrated in FIG. 2, the authentication method, such as ID and password, fingerprint, or facial image is recorded in the authentication method space 213 of the personal authentication information space 204, and the identification information (ID) uniquely identifying the user at the server 101 is recorded in the personal ID space 214 of the personal authentication information space 204, in the measurement data 200 stored in the server 101.

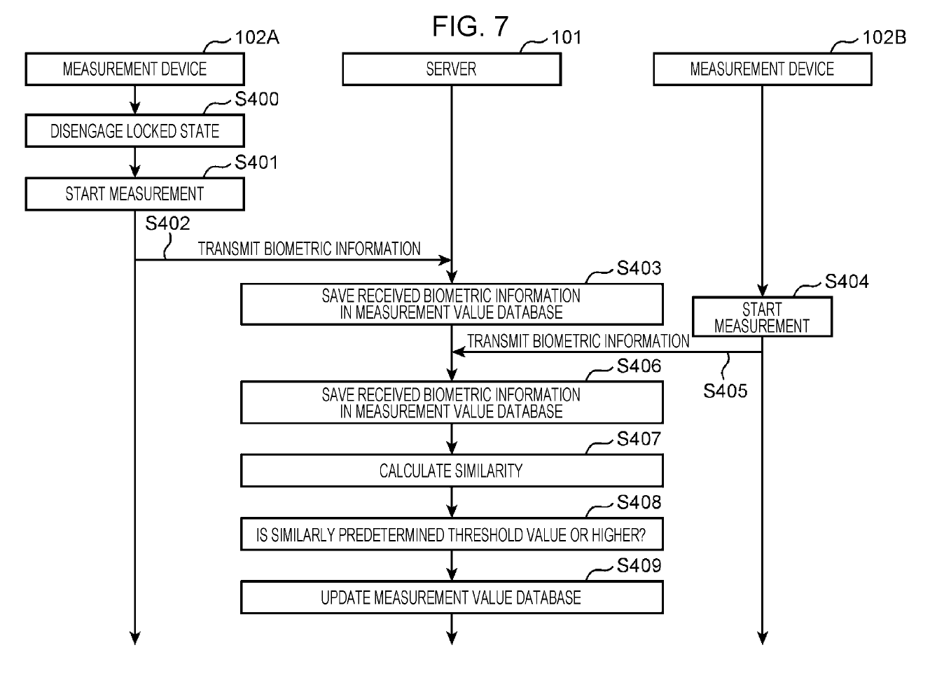

Next, a specific technique for succession of personal authentication information by comparing biometric information among multiple measurement devices will be described with reference to FIGS. 7 and 8. FIG. 7 is a sequence diagram schematically illustrating an example of personal authentication information succession procedures. In this first embodiment, the measurement device 102A (equivalent to an example of a first measurement device) has one or multiple types of personal authentication functions, out of functions of personal authentication by ID and password, functions of biometric personal authentication by fingerprint, and functions of biometric personal authentication by facial image. On the other hand, the measurement device 102B (equivalent to an example of a second measurement device) has no personal authentication functions. FIG. 7 illustrates an example of a specific sequence for the measurement device 102B to succeed personal authentication information of the measurement device 102A when the measurement device 102A and measurement device 102B transmit biometric information measurement data to the server 101. The measurement device 102A that has personal authentication functions will be understood to be in an unlocked state in step S400.

In the unlocked state in step S400, the measurement device 102A starts measurement of one or multiple types of biometric information of the user in step S401. Once measurement of biometric information is started in step S401, the measurement device 102A transmits measurement data (equivalent to an example of first vital data) of all biometric information measured at the measurement device 102A to the server 101 in step S402. In step S403, the server 101 records the measurement data of the biometric information that has been received in the personal authentication information space 204, which is saved in the measurement value database 111 within the server 101.

On the other hand, in step S404 the user starts measurement of one or multiple types of biometric information of the user using the measurement device 102B. Conceivable actions necessary for the user to start measurement of biometric information using the measurement device 102B include turning on the power of the measurement device 102B, the user wearing the measurement device 102B, the user entering a measurable range of the measurement device 102B, and so forth. The type of action is irrelevant here in the present embodiment, as long as it is an action whereby measurement of biometric information of the user is started using the measurement device 102B.

Upon measurement of biometric information of the user being started in step S404 using the measurement device 102B, biometric information measurement data (equivalent to an example of second vital data) that has been measured is transmitted from the measurement device 102B to the server 101 in step S405. In step S406, the server 101 saves the received biometric information measurement data in the measurement value database 111 of the server 101, in a state of not recording in the personal authentication information space 204.

In step S407, the similarity verifying unit 106 of the server 101 calculates the similarity between the features of the measurement data of the measurement device 102A and the features of the measurement data of the measurement device 102B, stored in the measurement value database 111 of the server 101.

The similarity verifying unit 106 uses measurement data of the same measurement type recorded in the type-of-measurement space 211 (FIG. 2) of the measurement value database 111. The similarity verifying unit 106 also uses measurement data taken in the same time slot. That is to say, the similarity verifying unit 106 uses measurement data of the measurement device 102A taken during the one minute of 12:00 to 12:01 and measurement data of the measurement device 102B taken during the one minute of 12:00 to 12:01, for example. Note that this is not restricted to one minute, and that the similarity verifying unit 106 may use, for example, measurement data taken during the five minutes of 12:00 to 12:05. This duration may be set beforehand for each measurement type, and the duration and measurement type may be stored in a correlated manner beforehand, in the ROM of the memory 108 for example.

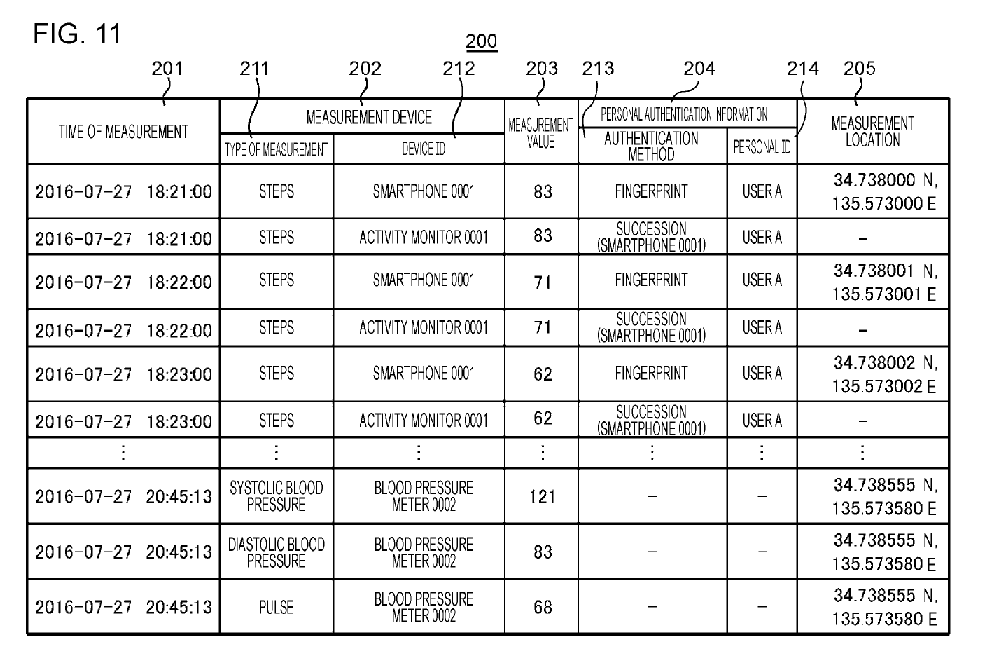

In step S408, the similarity verifying unit 106 determines whether or not the similarity calculated in step S407 is at a predetermined first threshold value or higher. In a case where determination is made that the similarity calculated in step S407 is the predetermined first threshold value or higher in step S408, in step S409 the similarity verifying unit 106 updates the measurement data 200 stored in the measurement value database 111 of the server 101 so that there is explicit indicating that the measurement device 102B regarding which similarity has been calculated in step S407 has succeeded the personal authentication information of the measurement device 102A. The measurement data 200 after updating will be described later with reference to FIG. 11.

FIG. 8 is a flowchart schematically illustrating an example of operation procedures of the similarity verifying unit 106 of the server 101, in the operations in FIG. 7. When the communication processing unit 103 starts to receive biometric information measurement data from the personal-authenticated measurement device 102A in step S411, and starts to save the received biometric information measurement data in the measurement value database 111 (step S403 in FIG. 7), the processing in FIG. 8 is started.

In step S412, the similarity verifying unit 116 extracts, out of the measurement data 200 stored in the measurement value database 111 of the server 101, data starting with the current point in time and going back a predetermined amount of time, based on the time-of-measurement space 201. The similarity verifying unit 106 determines in step S413 whether or not there is data of the measurement device 102B existing in the data that has been extracted in step S412. In a case where no data of the measurement device 102B exists in the extracted data (NO in step S413), the flow returns to step S412, and the similarity verifying unit 106 repeats extraction of data.

On the other hand, in a case where data of the measurement device 102B exists in the extracted data (YES in step S413), in step S414 the similarity verifying unit 106 separates the extracted data into data of the measurement device 102A and data of the measurement device 102B. The similarity verifying unit 106 extracts features of the measurement data from the measurement device 102A in step S415, and extracts features of the measurement data from the measurement device 102B in step S416.

Various techniques can be conceived for extracting features of the measurement data in steps S415 and S416, such as

(A1) a technique where the values themselves of the measurement data are taken as features,

(A2) a technique where the measurement data is subjected to Fourier transform, and frequency components are taken as features,

(A3) a technique where the measurement data is subjected to application of filters, and the resultant data is taken as features,

(A4) a technique where the measurement data is subjected to principal component analysis, and the obtained principal component is taken as features, and

(A5) a technique where one or a combination of multiple types of the techniques exemplified above is taken as features, but techniques for extracting features are not restricted to the above examples.

In step S417, the similarity verifying unit 106 calculates the similarity between the features of measurement data from the measurement device 102A and the features of measurement data from the measurement device 102B. Examples of techniques for calculating similarity in step S417 include

(B1) a technique where the proportion of the features of the measurement device 102A as to the features of the measurement device 102B is taken as the similarity,

(B2) a technique where the agreement rate of the features is taken as the similarity,

(B3) a technique where a correlation coefficient of the features is taken as the similarity,

(B4) a technique where a Pearson correlation of the features is taken as the similarity,

(B5) a technique where a proportion of error of the features is taken as the similarity, and

(B6) a technique where one or a combination of multiple types of the similarity calculation techniques exemplified above is taken as similarity, but techniques for calculating similarity are not restricted to the above examples.

Conceivable types of measurement data regarding which similarity can be calculated include blood pressure, number of steps walked, pulse, heart rate, electrocardiogram, weight, respiratory rate, depth of sleep, and so forth, and combinations thereof can be used as measurement data regarding which the similarity can be calculated, but the types of measurement data regarding which the similarity can be calculated are not restricted to the types exemplified here. The predetermined first threshold value can be decided beforehand in accordance with the type of similarity to be calculated, and saved in the memory 108.

In step S418, the similarity verifying unit 106 determines whether the similarity calculated in step S417 is at or above the predetermined first threshold value or not. In a case where the similarity is smaller than the predetermined first threshold value (NO in step S418), the flow returns to step S412, and the similarity verifying unit 106 resumes the flow from data extraction.

In a case where the set of measurement devices of which the similarity is at the predetermined first threshold value or higher has been uniquely identified (YES in step S418), the similarity verifying unit 106 updates the measurement value database 111 in step S419. An example of the updating will be described later with reference to FIG. 11.

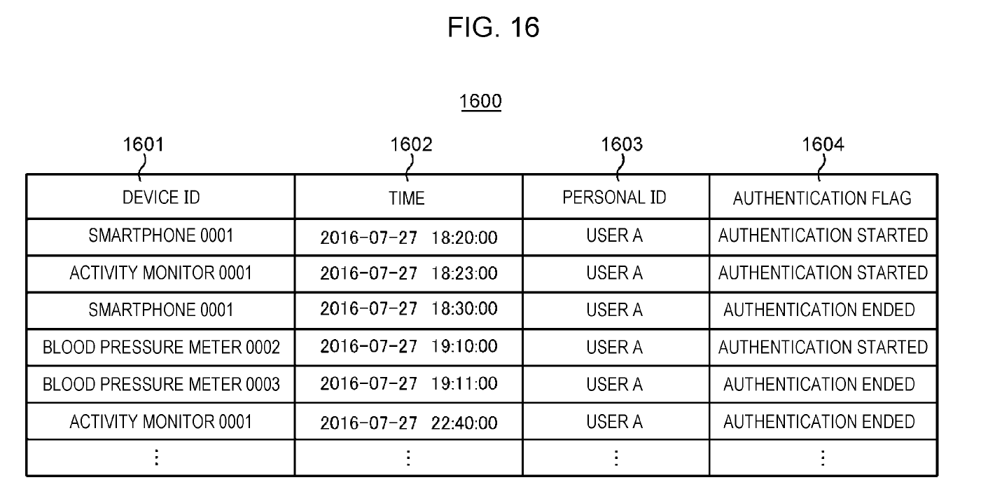

In step S420, the device management unit 105 registers the device ID of the measurement device 102B, the time, and so forth, in the authentication management database 113 of the server 101, to the effect that the measurement device 102B is a personal-authenticated device at the current point in time of step S412. The content of registration in the authentication management database 113 will be described later with reference to FIG. 16. After step S420, the flow returns to step S412, and the similarity verifying unit 106 resumes the flow of data extraction.

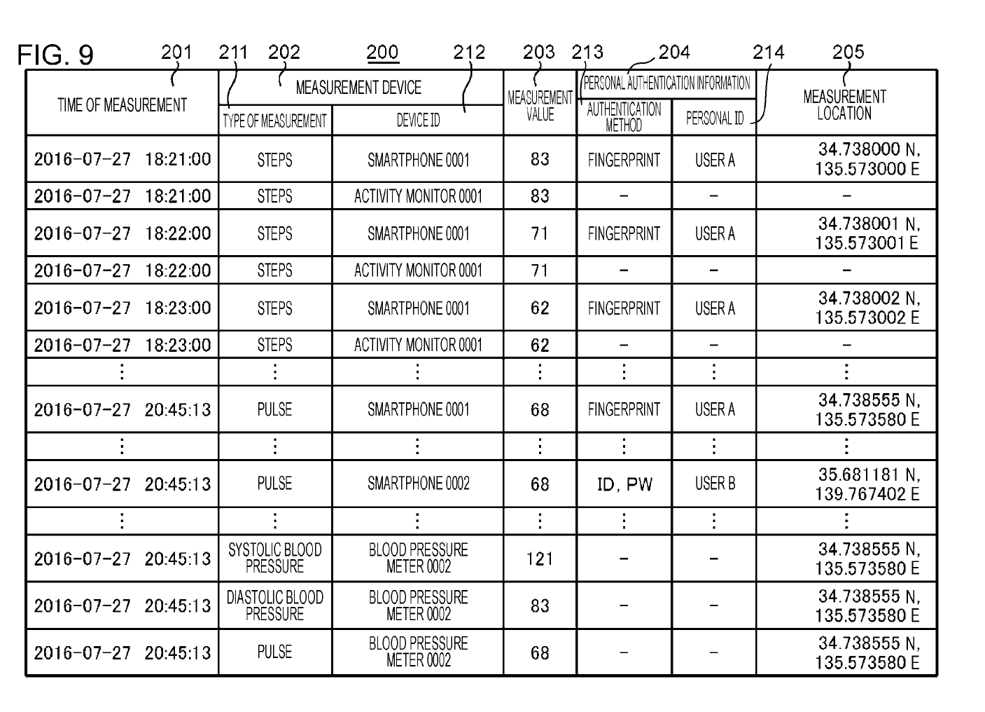

Now, a case where no measurement device where similarity is the threshold value or higher has been uniquely identified, but rather multiple are found in step S418, will be described. FIG. 9 is a diagram schematically illustrating an example of the measurement data 200 stored in the measurement value database 111 that is different from that in FIG. 2. There may be cases where, as illustrated in FIG. 9, the similarity between the features of “pulse” measured by “blood pressure meter 0002” and the features of “pulse” measured by “smartphone 0001” of “user A” is the threshold value or higher, and the similarity between the features of “pulse” measured by “blood pressure meter 0002” and the features of “pulse” measured by “smartphone 0002” of “user B” is the threshold value or higher.

In this case, the similarity verifying unit 106 compares the measurement location space 205 in the measurement value database 111. In the example in FIG. 9, the latitude and longitude recorded in the measurement location space 205 corresponding to the “blood pressure meter 0002” and the latitude and longitude recorded in the measurement location space 205 corresponding to the “smartphone 0001” agree. Thus, the similarity verifying unit 106 uniquely identifies the set of measurement devices of which the similarity is the threshold value or higher, by verifying that the measurement is being performed within a predetermined range.

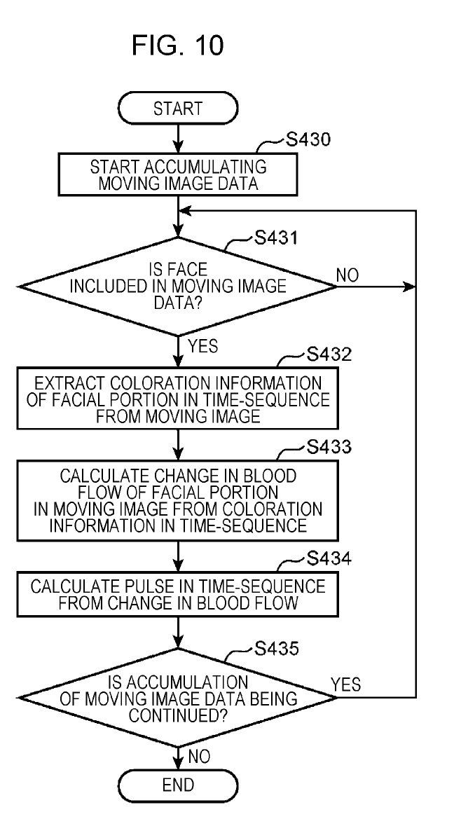

Now, a method of obtaining the pulse count by analyzing coloration information of the body surface in time sequence from a moving image including the face, in a case where the measurement device 102A has a camera, will be described. FIG. 10 is a flowchart schematically illustrating an example of procedures of the processing unit of the measurement device 102A that has a camera detecting pulse from a moving image.

The processing unit of the measurement device 102A starts accumulation of moving image data in step S430. In step S431, the processing unit determines whether or not a face is included in the moving image data. In step S432, the processing unit extracts coloration information of the facial portion of the moving image in time-sequence. In step S433, the processing unit calculates change in blood flow of the facial portion of the moving image, from coloration information in time sequence. In step S434, the processing unit calculates pulse in time sequence from the change in blood flow, and calculates the pulse count from the time intervals between pulses. In step S435, the processing unit determines whether or not accumulation of moving image data is continuing. If accumulation of moving image data is continuing (Yes in step S435), the flow returns to step S431, and the above-described steps are repeated. On the other hand, if accumulation of moving image data is not continuing (NO in step S435), the operations of FIG. 10 end.

FIG. 11 is a diagram illustrating a specific example of the update of the measurement value database 111 updated when there is succession of personal authentication information. Updating of the measurement value database 111 is performed in step S409 in FIG. 7 and step S419 in FIG. 8.

There is no succession of personal authentication information performed in the measurement data 200 described in FIG. 2. In comparison with this, the measurement data 200 illustrated in FIG. 11 has “succession (measurement device 102A)” indicating that personal authentication information has been succeeded recorded in the authentication method space 213 of the personal authentication information space 204. The measurement device 102A in the example in FIG. 11 is “smartphone 0001”. The ID recorded in the personal ID space 214 of the measurement device 102A, which is the source of succession of personal authentication information, is also recorded in the personal ID space 214 of the personal authentication information space 204.

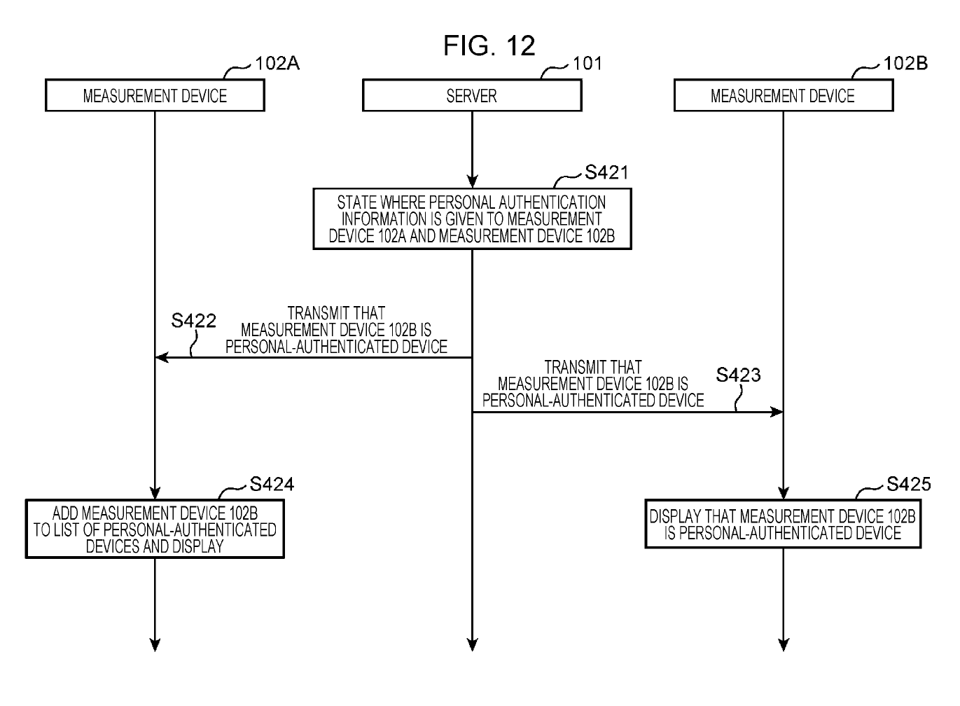

FIG. 12 is a sequence diagram schematically illustrating procedures for notifying that the measurement device 102B is to be handled as a personal-authenticated device. At the time of step S420 being executed in FIG. 8, the server 101 transmits the fact that the measurement device 102B is being handled as a personal-authenticated device to the measurement device 102A and the measurement device 102B by the procedures illustrated in FIG. 12.

In the state in step S421 in FIG. 21, personal authentication information is imparted to both the biometric information measurement data of the measurement device 102A in the measurement value database 111 of the server 101, and the biometric information measurement data of the measurement device 102B, at the current point in time at the server 101. When in the state in step S421, in step S422 the device management unit 105 of the server 101 transmits a signal to the measurement device 102A via the communication processing unit 103, representing that the measurement device 102B is a personal-authenticated device. When in the state in step S421, the device management unit 105 transmits a signal to the measurement device 102B via the communication processing unit 103 in step S423, representing that the measurement device 102B is a personal-authenticated device.

In step S424, the measurement device 102B is additionally displayed in the list of personal-authenticated devices displayed on the display unit of the measurement device 102A. In step S425, the fact that the measurement device 102B is a personal-authenticated device is displayed on the display unit of the measurement device 102B.

Forms of displaying on the display unit of the measurement device 102B that the measurement device 102B is a personal-authenticated device may conceivably include displaying on the display in text characters, displaying a mark indicating that a personal-authenticated device, and so forth, in a case where the measurement device 102B has a display. In a case where the measurement device 102B has a light-emitting diode (LED), conceivable forms include lighting the LED using a specified color, causing the LED to blink at predetermined intervals, and so forth. Also, one or a combination of multiple types of the exemplified forms may be used to display that the measurement device 102B is a personal-authenticated device at the display unit, display forms are not restricted as long as the user can confirm that the measurement device 102B is a personal-authenticated device. In a case where the measurement device 102B does not have a display unit for displaying that the measurement device 102B is a personal-authenticated device, step S425 is not executed.

FIG. 13 is a diagram schematically illustrating an example of exchange of signals between the server 101 and the devices 102A and 102B, and displays on the measurement devices 102A and 102B, when the processing described in FIGS. 7, 8, and 12 is executed.

In FIG. 13, in state 411 the measurement device 102A is authenticated and measuring, and transmitting biometric information to the server 101. On the other hand, the measurement device 102B is unauthenticated and not measuring. This state 411 transitions to a state 412 where the measurement device 102B has started measurement. In state 412, in addition to the state where the measurement device 102A that had been measuring and continuing to transmit measurement data to the server 101 in step S402 (FIG. 7), the measurement device 102B transmits measurement data to the server 101 in step S405 (FIG. 7).

Upon step S405 being executed, the similarity between the measurement data of the measurement device 102A and the measurement data of the measurement device 102B is calculated at the server 101 (step S407 in FIG. 7), the similarity is compared with the threshold value (step S408 in FIG. 7), and determination is made regarding whether or not the measurement device 102B should be taken as being a personal-authenticated device. If determination is made at the server 101 that it is acceptable for the measurement device 102B to be taken as a personal-authenticated device, the fact that the measurement device 102B is a personal-authenticated device is transmitted to the measurement device 102A and the measurement device 102B (steps S422 and S423 in FIG. 12). Accordingly, the state transitions from state 412 to state 413, the measurement device 102B is added to the personal-authenticated device list on the display unit 102Ad of the measurement device 102A, and an LED 102Bd of the measurement device 102B is lit to indicate that it is a personal-authenticated device, and the measurement device 102B is in an authenticated and measuring state.

Although an example of a smartphone is illustrated in FIG. 13 regarding the measurement device 102A, the measurement device 102A is not restricted to being a smartphone. It is sufficient for the measurement device 102A to have personal authentication functions such as exemplified in FIGS. 3, 5, and 6, to have a function of communication with the server 101, and to have a display unit that can display information, and to have measurement functions for measuring one or more biometric information of blood pressure, number of steps walked, pulse count, heart rate, electrocardiogram, weight, respiratory rate, and depth of sleep. In the first embodiment, blood pressure, the number of steps walked, pulse count, heart rate, electrocardiogram, weight, respiratory rate, and depth of sleep, are equivalent to an example of measurement types.

Although an example of a wristwatch-type activity monitor is illustrated in FIG. 13 regarding the measurement device 102B, the measurement device 102B is not restricted to being a wristwatch-type activity monitor. It is sufficient for the measurement device 102B to have functions of communicating with the server 101, and to have measurement functions for measuring measurement items of blood pressure, number of steps walked, pulse count, heart rate, electrocardiogram, weight, respiratory rate, and depth of sleep, that the measurement device 102A also is capable of measuring. Other conceivable specific examples of the measurement device 102B include a blood pressure meter, body composition meter, bathroom scales, sleep monitor, activity monitor other than a wristwatch-type, and so forth. The measurement device 102B may also analyze coloration information of the body surface from a moving image including the face to obtain the pulse count, as described with reference to FIG. 10.

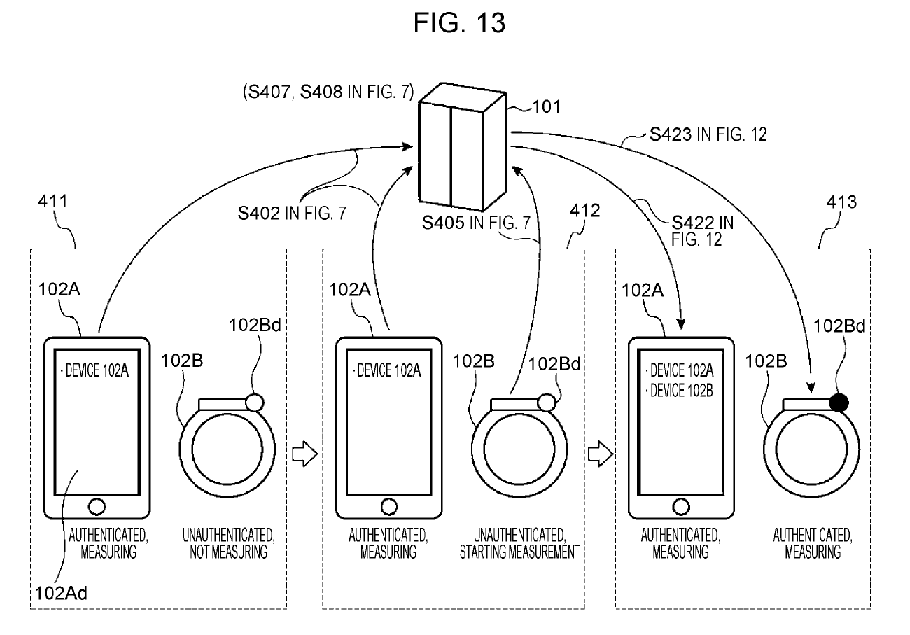

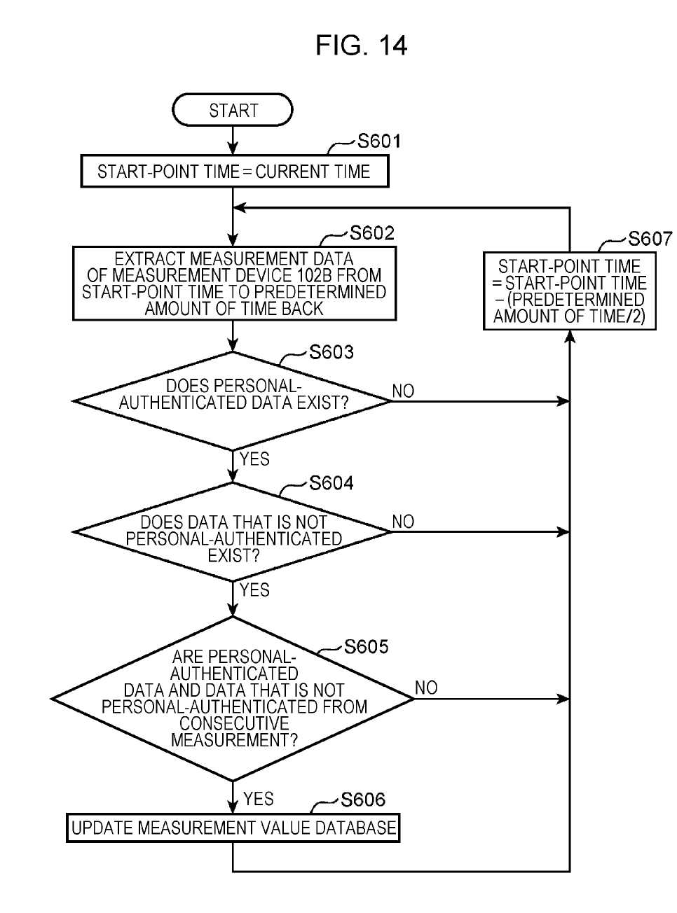

FIG. 14 is a flowchart schematically illustrating an example of other procedures for imparting personal authentication information to the measurement data of the measurement device 102B. According to the procedures in FIG. 14, in a case where succession of personal authentication information is performed by comparing the biometric information between the measurement device 102A and measurement device 102B, as described in FIGS. 7 and 8, even if the similarity of measurement data between the measurement device 102A and measurement device 102B cannot be calculated, personal authentication information can be imparted to the measurement data of the measurement device 102B using continuity of measurement data. The operations of FIG. 14 are executed while reception of measurement data from the measurement device 102B is continuing, every predetermined amount of time for example.

In step S601, the determining unit 104 sets the start-point time to the current time at the server 101. In step S602, the determining unit 104 extracts measurement data of the measurement device 102B from the start-point time to a predetermined amount of time back, from the measurement value database 111.

In step S603, the determining unit 104 determines whether or not data to which personal authentication information has been imparted exists in the data extracted in step S602. In a case where no data to which personal authentication information has been imparted exists in the data extracted in step S602 (NO in step S603), the flow advances to step S607. On the other hand, in a case where data to which personal authentication information has been imparted exists in the data extracted in step S602 (YES in step S603), the processing advances to step S604. In step S607, the determining unit 104 updates the start-point time to a point of time in the past that is half the predetermined time, and thereafter the flow returns to step S602.

In step S604, the determining unit 104 determines whether or not data to which personal authentication information has not been imparted exists in the data extracted in step S602. In a case where no data to which personal authentication information has not been imparted exists in the data extracted in step S602 (NO in step S604), the flow advances to step S607. On the other hand, in a case where data to which personal authentication information has not been imparted exists in the data extracted in step S602 (YES in step S604), the processing advances to step S605. In step S605, the determining unit 104 determines whether or not the data to which personal authentication information has been imparted and the data to which personal authentication information has not been imparted in the extracted measurement data of the measurement device 102B are from consecutive measurement by the measurement device 102B.

Conceivable determination techniques, for determining whether or not consecutive measurement, include determining not to be consecutive measurement in

(C1) a case where data explicitly indicating end of measurement is recorded between the data to which personal authentication information has been imparted and the data to which personal authentication information has not been imparted, and

(C2) a case where the greatest value of an interval between data to which personal authentication information has been imparted and the data to which personal authentication information has not been imparted exceeds a predetermined threshold value.

Techniques where the exemplified consecutive measurement determination techniques are combined are also conceivable. Note that it is sufficient to be able to determine whether or not consecutive measurement, and the determining techniques are not restricted.

In a case where it has been found in step S605 that data to which personal authentication information has been imparted and the data to which personal authentication information has not been imparted of the measurement data of the measurement device 102B was due to consecutive measurement by the measurement device 102B (YES in step S605), the flow advances to step S606. On the other hand, in a case where it has been found that data to which personal authentication information has been imparted and the data to which personal authentication information has not been imparted of the measurement data of the measurement device 102B was not due to consecutive measurement by the measurement device 102B (NO in step S605), the flow advances to step S607.

In step S606, the determining unit 104 imparts personal authentication information to the measurement data not imparted with personal authentication information, and updates the measurement value database 111 of the server 101. Thereafter, the flow advances to step S607.

Although the start-point time is extended back in time by half of the predetermined time, half of the predetermined time is not restrictive, and it is sufficient to have overlapping in the data being extracted. An arrangement may be made where, when the start-point time reaches the initial measurement time when start-point time is extended back, the operations of FIG. 14 end.

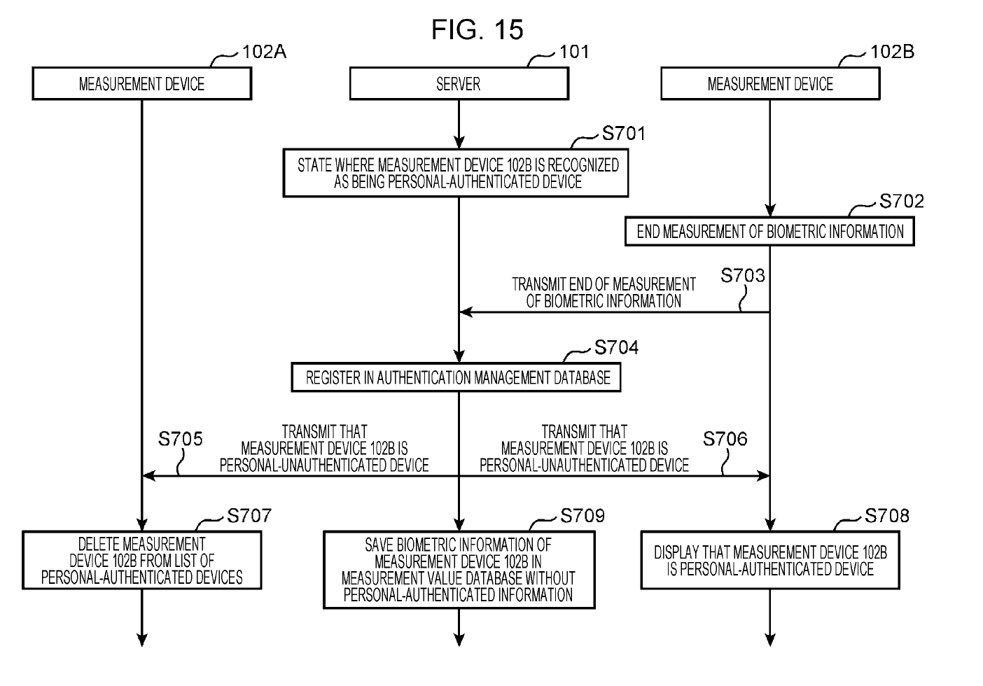

FIG. 15 is a sequence diagram schematically illustrating operations in a case of the measurement device 102B that has succeeded personal authentication information from the measurement device 102A having personal authentication functions ends measurement of biometric information. The operations in FIG. 15 are executed in a state where the measurement device 102B is recognized as being a personal-authenticated device (step S701).

Upon the measurement device 102B having ended measurement of biometric information in step S702, in step S703 the measurement device 102B transmits an end to measurement of biometric information to the server 101. In step S704, the device management unit 105 of the server 101 registers in the authentication management database 113 (FIG. 16) that handling the measurement device 102B as a personal-authenticated device has been stopped. Accordingly, the measurement data of biometric information transmitted from the measurement device 102B thereafter is saved in the measurement value database 111 without having personal authentication information imparted thereto.

In step S705, the device management unit 105 of the server 101 transmits to the measurement device 102A that the measurement device 102B is a personal-unauthenticated device. In step S706, the device management unit 105 transmits to the measurement device 102B that the measurement device 102B is a personal-unauthenticated device. In step S707, the measurement device 102A deletes the measurement device 102B from the personal-authenticated device list on the display unit 102Ad (FIG. 13) of the measurement device 102A, and updates the personal-authenticated device list and displays. In step S708, the measurement device 102B displays that the measurement device 102B is a personal-unauthenticated device by turning off the LED 102Bd (FIG. 13). In a case where the measurement device 102B does not have a display unit such as an LED or the like to display that it is a personal-unauthenticated device, step S708 is not executed.

Regarding biometric information measurement data of the measurement device 102B received from step S704 and thereafter, in step S709, the server 101 saves the biometric information of the measurement device 102B in the measurement value database 111 without authentication information, until biometric information measurement data is transmitted from the measurement device 102A and comparison of similarity with the biometric information of the measurement device 102B is performed, to deem to be personal-authenticated.

FIG. 16 is a diagram schematically illustrating an example of authentication management information 1600 stored in the authentication management database 113. The authentication management information 1600 has a device ID space 1601, time space 1602, personal ID space 1603, and authentication flag space 1604, as illustrated in FIG. 16. Information the same as that in the device ID space 212 and personal ID space 214 of the measurement data 200 (FIG. 2) is respectively stored in the device ID space 1601 and personal ID space 1603. The time of the device management unit 105 starting or ending personal authentication is recorded in the time space 1602. In a case of starting personal authentication, a flag indicating “authentication started” is recorded in the authentication flag space 1604, and in a case of ending personal authentication, a flag indicating “authentication ended” is recorded. Reading out information recorded in the authentication management information 1600 enables the state of personal authentication measurement devices recorded in the device ID space 1601 to be comprehended.

### Second Embodiment

An example of the measurement device 102B not having personal authentication functions succeeding personal authentication information of the measurement device 102A having personal authentication functions has been described in the first embodiment. In a second embodiment, an example will be described where the measurement device 102C (equivalent to an example of a third measurement device) that does not have personal authentication functions further succeeds the personal authentication information that the measurement device 102B (equivalent to an example of a second measurement device) that does not have personal authentication functions has succeeded.

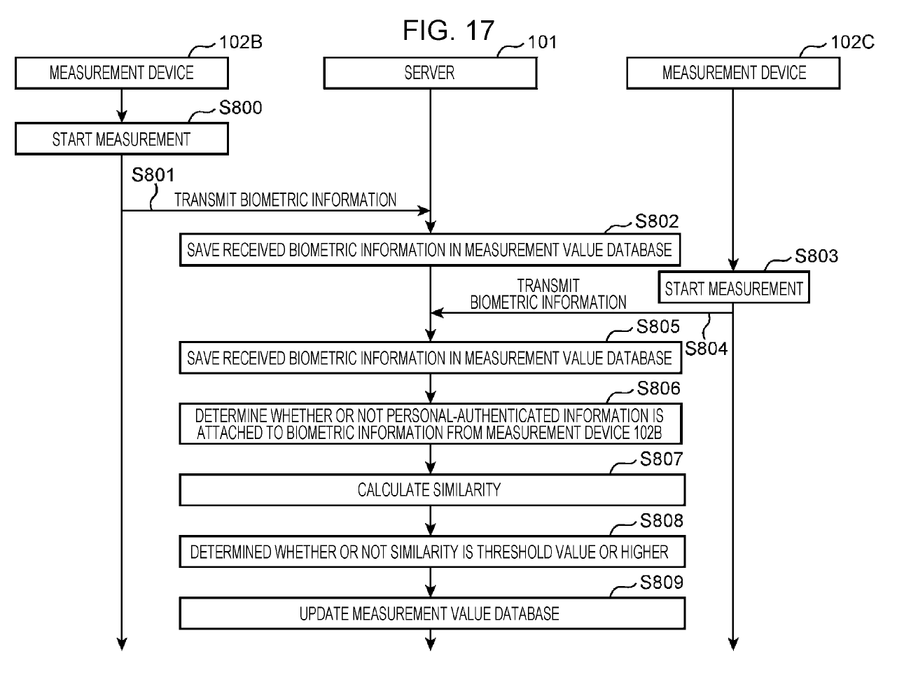

FIG. 17 is a sequence diagram schematically illustrating an example of succession procedures of personal authentication information in the second embodiment. In step S800, the measurement device 102B starts measurement of one or multiple types of biometric information of the user. Upon step S800 being executed, the measurement device 102B transmits measurement data (equivalent to an example of second vital data) of all biometric information measured at the measurement device 102B to the server 101 in step S801. In step S802, the server 101 saves the measurement data of the biometric information that has been received from the measurement device 102B in the measurement value database 111 within the server 101. On the other hand, the measurement device 102C starts measurement of one or multiple types of biometric information of the user in step S803.

Conceivable user actions necessary for the measurement device 102B and measurement device 102C to start measurement of biometric information in steps S801 and S803 include

(D1) turning on the power of the measurement device 102B and measurement device 102C,

(D2) the user wearing the measurement device 102B and measurement device 102C,

(D3) the user entering a measurable range of the measurement device 102B and measurement device 102C,

and so forth. The type of action is irrelevant, as long as it is an action whereby measurement of biometric information of the user is started using the measurement device 102B and measurement device 102C. The measurement at the measurement device 102B and measurement device 102C does not have to be started by the same action.

Upon step S803 having been executed, the measurement device 102C transmits all biometric information measurement data (equivalent to an example of third vital data) that has been measured by the measurement device 102C to the server 101 in step S804. In step S805, the server 101 saves the biometric information measurement data received from the measurement device 102C in the measurement value database 111 in the server 101.

In step S806, the device management unit 105 of the server 101 determines whether or not personal authentication information has been imparted to the measurement data from the measurement device 102B that is stored in the measurement value database 111. In the case in FIG. 2 for example, the personal authentication information space 204 corresponding to the “activity monitor 0001” in the device ID space 212 is blank, so the device management unit 105 determines that no personal authentication information is imparted to the measurement data from the activity monitor 0001 (example of measurement device 102B). In the case in FIG. 11 for example, it is recorded in the personal authentication information space 204 corresponding to the “activity monitor 0001” in the device ID space 212 that personal authentication information has been succeeded, so the device management unit 105 determines that personal authentication information has been imparted to the measurement data from the activity monitor 0001 (example of measurement device 102B).

In a case where no personal authentication information has been imparted to the measurement data from the measurement device 102B that is stored in the measurement value database 111 of the server 101 in step S806, subsequent processing is not performed, and the flow returns to step S805. In a case where personal authentication information has been imparted to the measurement data from the measurement device 102B that is stored in the measurement value database 111 of the server 101 in step S806, the flow advances to step S807. In step S807, the similarity verifying unit 106 of the server 101 calculates the similarity (equivalent to second value indicating degree of agreement) between features of the measurement data of the measurement device 102B stored in the measurement value database 111 of the server 101 and features of the measurement data of the measurement device 102C.

The similarity verifying unit 106 uses measurement data of the same measurement type recorded in the type-of-measurement space 211 (FIG. 2) of the measurement value database 111 at this time. That is to say, the similarity verifying unit 106 uses measurement data taken in the same time slot. That is to say, the similarity verifying unit 106 uses measurement data of the measurement device 102B taken during the one minute of 12:00 to 12:01 and measurement data of the measurement device 102C taken during the one minute of 12:00 to 12:01. Note that this is not restricted to one minute, and that the similarity verifying unit 106 may use, for example, measurement data taken during the five minutes of 12:00 to 12:05. This duration may be set beforehand for each measurement type, and the duration and measurement type may be stored in a correlated manner beforehand, in the ROM of the memory 108 for example.

The techniques for similarity calculation performed in step S807 may conceivably be the techniques (B1) through (B6) exemplified in the first embodiment but the techniques for calculating similarity are not restricted to the examples, as in the first embodiment. The predetermined second threshold value can be decided beforehand in accordance with the type of similarity to be calculated, and saved in the memory 108.

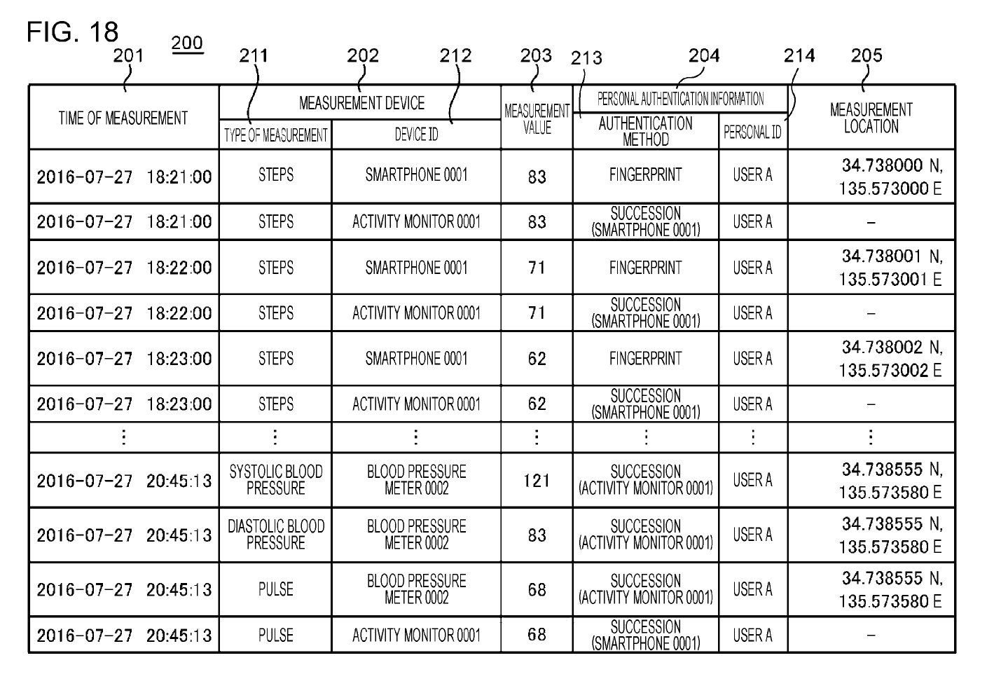

In step S808, the similarity verifying unit 106 determines whether or not the similarity calculated in step S807 is at a predetermined second threshold value or higher. In a case where determination is made that the similarity calculated in step S807 is the predetermined second threshold value or higher in step S808, step S809 is executed. In step S809, the similarity verifying unit 106 updates the measurement data 200 that is stored in the measurement value data base 111 of the server 101, so that there is explicit indication that the measurement device 102C regarding which similarity has been calculated in step S807 has succeeded the personal authentication information of the measurement device 102B, as illustrated in FIG. 18. Specific processing performed in steps S807, S808, and S809 can be realized by techniques described with regard to FIG. 8.

FIG. 18 is a diagram illustrating a specific example of the measurement value database 111 updated in step S809 in FIG. 17. In the example in FIG. 18, out of the IDs recorded in the device ID space 212, the measurement device 102B is the “activity monitor 0001”, and the measurement device 102C is the “blood pressure meter 0002”.

There is no succession of personal authentication information performed regarding the “blood pressure meter 0002” in device ID space 212 in the updated measurement data 200 described in FIG. 11. In comparison with this, the measurement data 200 illustrated in FIG. 18 has “succession (activity monitor 0001)” indicating that personal authentication information has been succeeded from the measurement device 102B (i.e., the activity monitor 0001) recorded in the authentication method space 213 of the personal authentication information space 204 in the “blood pressure meter 0002” in the device ID space 212. The ID recorded in the personal ID space 214 of the measurement device 102B (i.e., the activity monitor 0001), which is the source of succession of personal authentication information, is also recorded in the personal ID space 214 of the personal authentication information space 204, i.e., “user A”.

Note that pulse is measured by both the “activity meter 0001” that is an example of the measurement device 102B, and the “blood pressure meter 0002” that is an example of the measurement device 102C, as illustrated in the measurement data 200 in FIG. 18. Accordingly, the similarity can be calculated using the pulse measurement data in step S807 in FIG. 17.

### Third Embodiment

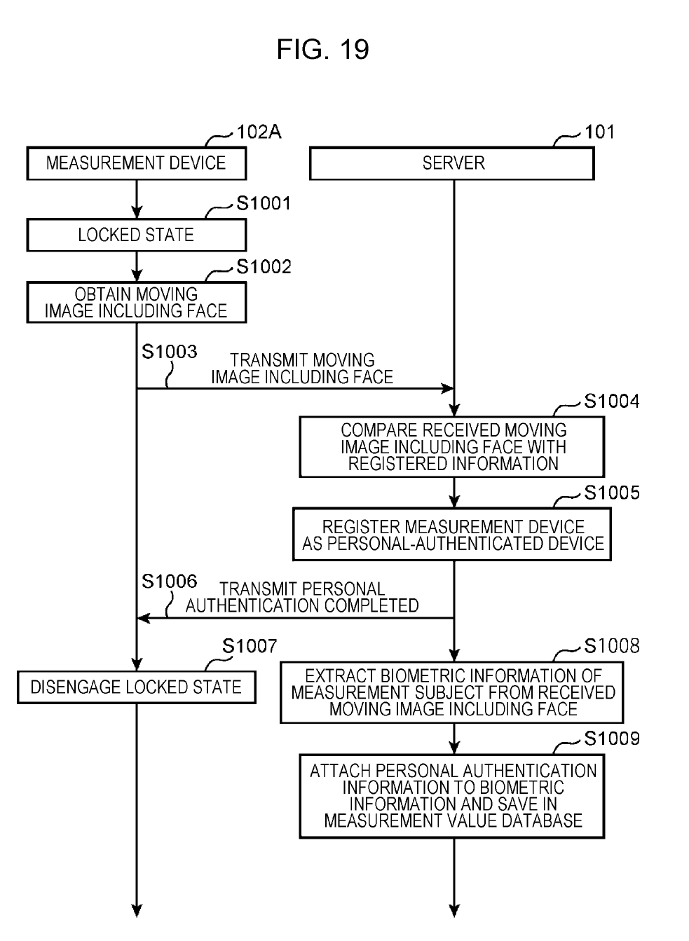

FIG. 19 is a sequence diagram schematically illustrating an example of procedures for imparting personal authentication information to biometric information measurement data that has been measured in a third embodiment. In the third embodiment, a personal authentication mechanism having personal authentication functions in the measurement device 102A that measures biometric information has functions of measuring biometric information. The measurement device 102A has a camera in the example in FIG. 19.

In step S1001, the measurement device 102A is in a state where personal authentication has not been performed and the device is locked. From this locked state, the measurement device 102A prompts the user to use the camera to shoot a moving image including the face of the user in step S1002. The measurement device 102A obtains a moving image including the face of the user that has been shot.

In step S1003, the moving image including the face of the user that has been shot is transmitted from the measurement device 102A to the server 101. The determining unit 104 of the server 101 compares the features of the received moving image with the personal authentication information recorded in the personal authentication database 112 of the server 101 in step S1004, and determines whether or not the face in the moving image is the face of a registered user. In a case where determination is made that the face in the moving image is not the face of the registered user, the following processing is not executed.

In a case where determination is made that the face in the moving image is the face of the registered user, the determining unit 104 of the server 101 registers the measurement device 102A as being a personal-authenticated device of the registered user in step S1005. In step S1006, transmission is made to the effect that the measurement device 102A has been registered as a personal-authenticated device to the measurement device 102A from the server 101. The locked state of the measurement device 102A is disengaged in step S1007.

In step S1008, the server 101 extracts features corresponding to coloration information from the moving image including the face of the user that has been received, and detects biometric information of the user. Biometric information that can be detected include the pulse count, heart rate, state of stress, respiratory count, and so forth, of the user. For example, the pulse count can be detected using the procedures described with reference to FIG. 10. Although one or multiple types of the examples of biometric information can be detected, the types of detectable biometric information are not restricted. In step S1009, the server 101 imparts personal authentication information of the registered user to the detected biometric information, and saves in the measurement value database 111 of the server 101.

Although an example where the personal authentication mechanism is a camera has been exemplified in FIG. 19, the personal authentication mechanism is not restricted to being a camera. For example, in a case where biometric information such as pulse count can be measured or extracted from the finger of the user using a fingerprint authentication mechanism, the measurement device 102A may have a fingerprint authentication mechanism as the authentication mechanism thereof. Further, the type of authentication performed by the authentication mechanism of the measurement device 102A is not restricted.

### Fourth Embodiment

A determining device that determines the state of measurement when measuring blood pressure using a blood pressure meter will be described in a fourth embodiment. A case where a wrist-worn blood pressure meter that is worn on the wrist to measure blood pressure will be described in the fourth embodiment.

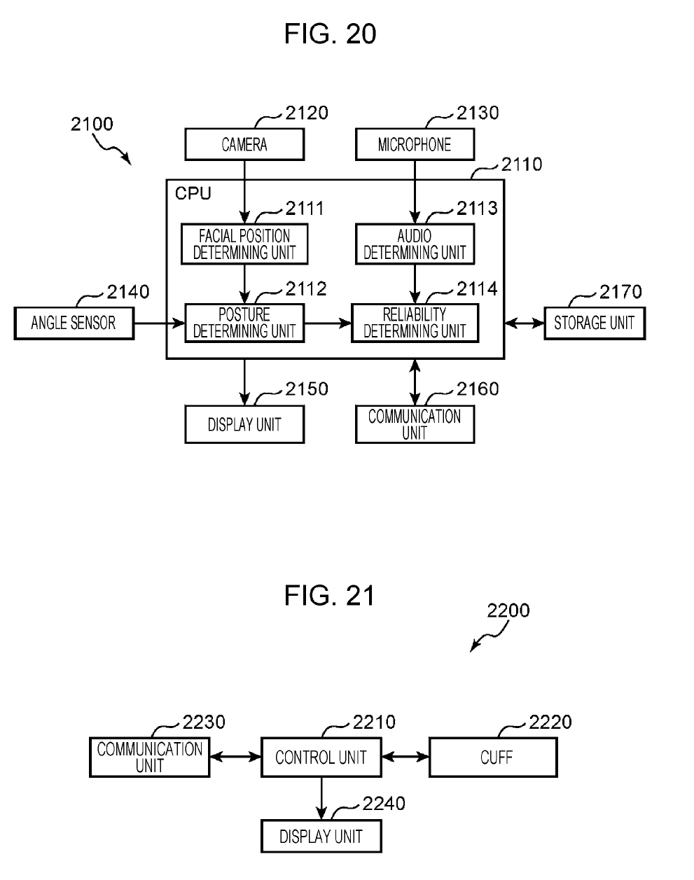

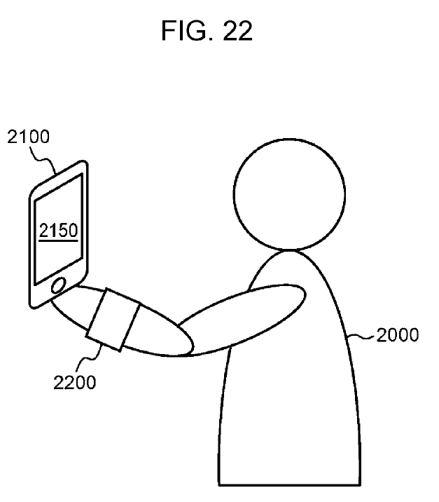

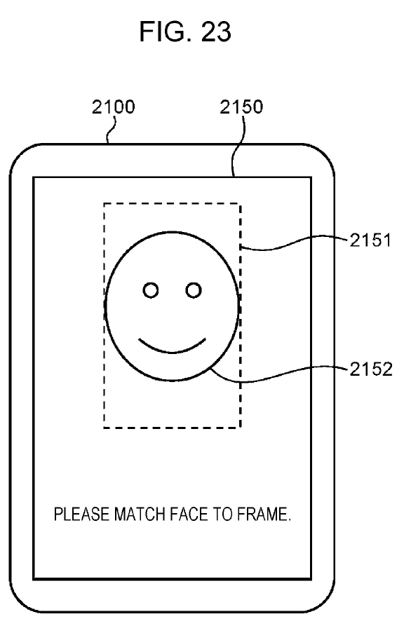

FIG. 20 is a block diagram schematically illustrating an example of the configuration of the determining device according to the fourth embodiment. FIG. 21 is a block diagram schematically illustrating an example of the configuration of a blood pressure meter. FIG. 22 is a diagram schematically illustrating a measurement state when a user uses the blood pressure meter to measure blood pressure. FIG. 23 is a diagram schematically illustrating an example of a screen displayed on a display unit of the determining device before starting measurement of blood pressure.

As illustrated in FIG. 20, the determining device 2100 according to the fourth embodiment includes a camera 2120, an angle sensor 2140, a microphone 2130, a display unit 2150, a CPU 2110, a communication unit 2160, and a storage unit 2170.

The storage unit 2170 is made up of semiconductor memory or the like, for example. The storage unit 2170 includes, for example, ROM, RAM, EEPROM, and so forth. The ROM of the storage unit 2170 stores a control program according to the fourth embodiment to operate the CPU 2110. The storage unit 2170 records information such as audio data, image data, blood pressure measurement values, and so forth. Although the storage unit 2170 is built into the determining device 2100 in the fourth embodiment, this is not restrictive. The storage unit 2170 may be memory in a server connected to a network, for example.

The CPU 2110 operates in accordance with the control program according to the fourth embodiment stored in the storage unit 2170, to function as a facial position determining unit 2111, a posture determining unit 2112, an audio determining unit 2113, and a reliability determining unit 2114.

The determining device 2100 is held by one hand or both hands of a user 2000 who has the blood pressure meter 2200 worn on a wrist, such that the camera 2120 takes images of the face of the user 2000, as illustrated in FIG. 22. In the fourth embodiment, the determining device 2100 is held by the one hand of the user 2000 on which the blood pressure meter 2200 is worn. A smartphone is used as an example of the determining device 2100 in the fourth embodiment. A tablet computer may be used as the determining device 2100 as well.

A blood pressure meter 2200 that interoperates with the determining device 2100 includes a control unit 2210, a cuff 2220, a communication unit 2230, and a display unit 2240, as illustrated in FIG. 21. The control unit 2210 includes a CPU, memory, and so forth, and performs computation processing or the like. The cuff 2220 is a bag-like belt. The cuff 2220 is attached to the wrist of the user 2000 holding the determining device 2100, for example, in the fourth embodiment. The communication unit 2230 performs communication with the determining device 2100. The display unit 2240 displays measurement results.

Next, the configurations of the determining device 2100 will be described. The camera 2120 shoots still or moving images. The camera 2120 shoots the face of the user 2000 and inputs image data to the CPU 2110.

The angle sensor 2140 obtains first information indicating the angle of inclination of the normal to the surface of the display unit 2150 of the determining device 2100 as to the gravitational direction. The angle sensor 2140 is configured of a gyroscope or acceleration sensor, for example. The angle sensor 2140 is provided to the determining device 2100 in the fourth embodiment. An angle sensor provided to the blood pressure meter 2200 may be used instead of the angle sensor 2140. In this case, reference data representing the correlation between the angle of the wrist and the angle of the determining device 2100 may be used.

The microphone 2130 obtains audio around the determining device 2100. The microphone 2130 inputs the obtained audio to the CPU 2110. The display unit 2150 is controlled by the CPU 2110 and displays messages to prompt the user 2000 to assume an appropriate posture, blood pressure measurement values, reliability of measurement values, and so forth. Specific examples of screens displayed on the display unit 2150 will be described later. The communication unit 2160 is controlled by the CPU 2110 and performs wired or wireless communication with the blood pressure meter 2200. The communication unit 2160 transmits an instruction to the blood pressure meter 2200 to start measurement, receives measurement results from the blood pressure meter 2200, and exchanges data through communication with an external server and so forth.

The facial position determining unit 2111 obtains second information indicating the position and size of the face within the image data, from the image data of the camera 2120 of the determining device 2100. The second information is used by the posture determining unit 2112 to determine posture. The audio determining unit 2113 compares the audio data of the microphone 2130 of the determining device 2100 with exhaust sound data of the cuff 2220 stored in the storage unit 2170 beforehand, and determines whether or not the exhaust sound of the cuff 2220 is included in the audio obtained by the microphone 2130.

The posture determining unit 2112 determines the height at which the cuff 2220 is situated, from the second information obtained from the facial position determining unit 2111 and the first information obtained from the angle sensor 2140. Specifically, the posture determining unit 2112 determines that the cuff 2220 is situated at the same height as the heart of the user 2000 in a case where the angle indicated by the first information is within a predetermined first range, the position of the face of the user 2000 within the image data indicated in the second information is within a predetermined second range, and the proportion of the size of the face of the user 2000 in the image data indicated by the second information is in a predetermined third range. The posture determining unit 2112 outputs to the display unit 2150, for example, that determination has been made that the cuff 2220 is situated at the same height as the heart of the user 2000.

The posture determining unit 2112 may determine that the angle indicated in the first information is within the predetermined first range if the difference between the angle of inclination of the normal to the surface of the display unit 2150 of the determining device 2100 as to the gravitational direction, decided beforehand so that the position of the cuff 2220 of the blood pressure meter 2200 will be at the same height as the heart of the user 2000, and the first information obtained by the angle sensor 2140 is at or below a threshold value decided beforehand. The storage unit 2170 may store beforehand the above-described angle of inclination of the normal to the surface of the display unit 2150 of the determining device 2100 as to the gravitational direction, decided beforehand so that the position of the cuff 2220 of the blood pressure meter 2200 will be at the same height as the heart of the user 2000.

When starting measurement of blood pressure, the posture determining unit 2112 displays a frame 2151 of a size decided beforehand at a position on the display unit 2150 decided beforehand, and displays an instruction to the user 2000, such as “please match face to frame”, below the frame 2151. The posture determining unit 2112 may determine that when a facial image 2152 of the user 2000 taken by the camera 2120 is situated within the frame 2151, the position of the face of the user 2000 within the image data indicated in the second information is within the predetermined second range, and the proportion of the size of the face of the user 2000 in the image data indicated by the second information is in the predetermined third range.

The size of the facial image 2152 (FIG. 23) of the user 2000 taken by the camera 2120 displayed on the display unit 2150 is dependent on the distance between the camera 2120 (i.e., the determining device 2100), and the face of the user 2000. Accordingly, the sideways length of the frame 2151 can be decided beforehand taking into consideration a general length of the arm and size of the face of the user 2000.

The height position of the facial image 2152 of the user 2000 displayed on the display unit 2150 is dependent on the angle of the user 2000 holding the determining device 2100, and the height of the determining device 2100. Accordingly, the length and position of the frame 2151 in the vertical direction, and the angle of the normal to the surface of the display unit 2150 of the determining device 2100 as to the gravitational direction described above, can be decided beforehand so that the height of the wrist of the user 2000 holding the determining device 2100 matches the height of the heart of the user 2000.

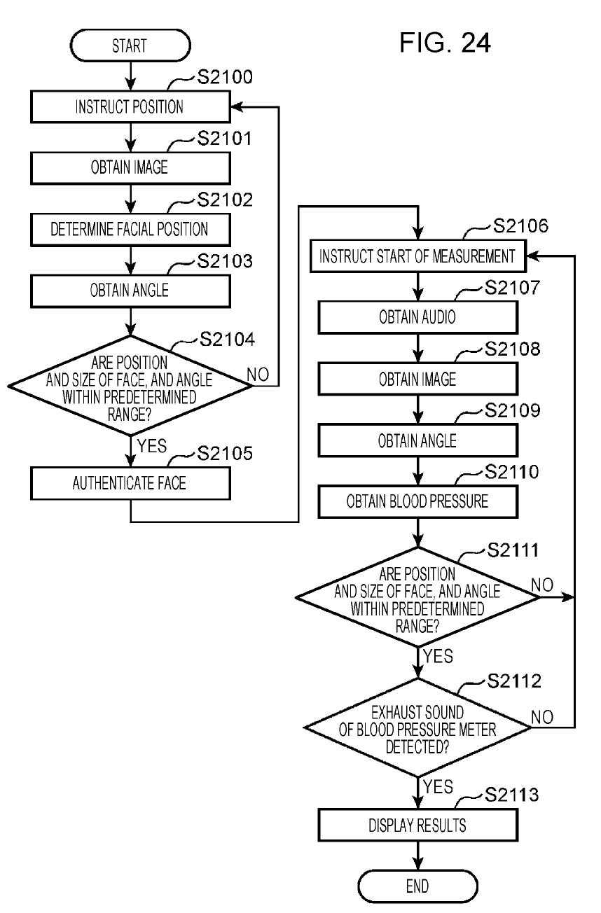

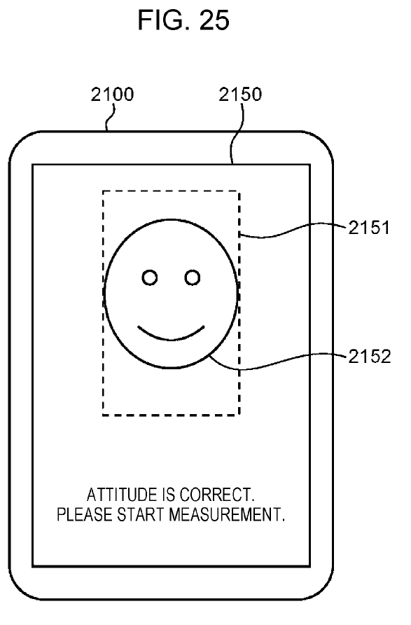

FIG. 24 is a flowchart schematically illustrating operations of the determining device 2100 when measuring blood pressure according to the fourth embodiment. FIGS. 25 through 28 are diagrams schematically illustrating examples of displays made at the determining device when measuring blood pressure. The operations of the determining device 2100 will be described with reference to FIGS. 23 through 28.

First, the user 2000 wears the cuff 2220 of the blood pressure meter 2200 on the arm. The blood pressure meter that is used may be of a type worn on the wrist or a type worn on the upper arm, but an example of a wrist-worn blood pressure meter will be described in the fourth embodiment. The user 2000 holds the determining device 2100 in one hand or both hands (in the fourth embodiment, the one hand at the side to which the blood pressure meter 2200 is worn, as described above), and directs the camera 2120 toward the face of the user 2000.

In step S2100, the posture determining unit 2112 prompts the user 2000 to assume a correct posture through the display unit 2150. In the fourth embodiment, the posture determining unit 2112 displays the frame 2151 and the facial image 2152 taken by the camera 2120 on the display unit 2150, as illustrated in FIG. 23, and instructs the user 2000 to move the hand holding the determining device 2100 so that the facial image 2152 is within the frame 2151. The method for presenting a correct posture is not restricted to this, and for example, the posture determining unit 2112 may simply display the direction in which the user 2000 should move the hand holding the determining device 2100 on the display unit 2150.

In step S2101, the facial position determining unit 2111 obtains an image including the face of the user 2000, taken by the camera 2120. In step S2102, the facial posture determining unit 2111 obtains the second information indicating the position and size of the face within the image from the obtained image, and hands the second information to the posture determining unit 2112. This position of the face is the center position of the region of the face of the user 2000 in the image data taken by the camera 2120, for example. The size of the face is, for example, information indicating the proportion of the area of the face of the user 2000 in the image data taken by the camera 2120.

In step S2103, the posture determining unit 2112 obtains first information indicating the angle of the normal to the surface of the display unit 2150 of the determining device 2100 as to the gravitational direction, from the angle sensor 2140. Thereafter, in step S2104, the posture determining unit 2112 determines whether the angle indicated by the first information, the position of the face of the user 2000 within the image data, and the proportion of the size of the face of the user 2000 in the image data, indicated by the second information, are each within the predetermined ranges. In a case where the angle indicated by the first information is within the first range, the position of the face of the user 2000 in the image data indicated by the second information is within the second range, and the proportion of the size of the face of the user 2000 in the image data indicated by the second information is within the third range (YES in step S2104), the posture determining unit 2112 determines that the height of the cuff 2220 and the height of the heart of the user 2000 agree, and notifies the reliability determining unit 2114 to the effect that the user 2000 is using the blood pressure meter 2200 in a correct posture.

In a case of shooting the face of the user 2000 by the camera 2120 of the determining device 2100 held in the hand at the same side as the arm where the cuff 2220 is worn, the positional relations between the camera 2120, cuff 2220, face of user 2000, and heart are restricted. Accordingly, the relative position between the cuff 2220 and the heart of the user 2000 can be calculated from the position of face and proportion of size of face of the user 2000 in the image data taken by the camera 2120, indicated by the second information.

Further, the vertical relationship between the cuff 2220 and the heart can be determined by using the angle of the normal to the surface of the display unit 2150 of the determining device 2100 as to the gravitational direction, indicated by the first information obtained from the angle sensor 2140. The cuff 2220 and the heart are preferably at the same height, for correct blood pressure measurement. Accordingly, the posture can be determined by storing the angle detected by the angle sensor 2140 in a case where the cuff 2220 and the heart are at the same height, and the position of the face in the image and the proportion of the size of the face in the image, in the storage unit 2170, and performing determination that each are at predetermined values.

In a case where the position of the face, proportion of the size of the face, or the angle are not in the predetermined ranges, the determination results by the facial position determining unit 2111 and posture determining unit 2112 are preferably fed back to the 2000 user by the display unit 2150 as illustrated in FIG. 23, thereby prompting the user 2000 to assume a correct posture.

In step S2105, the facial position determining unit 2111 uses the image obtained in step S2101 to authenticate the face of the user 2000. The storage unit 2170 stores features of the facial image of the user 2000 beforehand. The facial position determining unit 2111 extracts features of the facial image of the user 2000 from the image obtained in step S2101. The facial position determining unit 2111 compares the extracted features and the features of the facial image of the user 2000 stored in the storage unit 2170 beforehand, and determines whether matching or not. By performing this personal authentication in step S2105, the person currently using the blood pressure meter 2200 can be identified, thereby preventing blood pressure measurement by another person.

In a case where personal authentication cannot be performed in step S2105, the blood pressure measurement may be terminated. Also, in a case where determination is made by the posture determining unit 2112 that the cuff 2220 is at a predetermined height (YES in step S2104), step S2105 may be skipped and the flow directly advance to step S2106.

In step S2106, the posture determining unit 2112 instructs starting of blood pressure measurement by the blood pressure meter 2200 via the communication unit 2160. The method of instructing starting of blood pressure measurement at this time may be to connect the communication unit 2160 and the blood pressure meter 2200 by cable or wirelessly, and instruct starting of blood pressure measurement via the communication unit 2160. Alternatively, the user 2000 may be instructed to start blood pressure measurement via the display unit 2150, as illustrated in FIG. 25, so that the user 2000 operates the blood pressure meter 2200.

After starting blood pressure measurement, in step S2107 the audio determining unit 2113 detects the exhaust sound of the cuff 2220 from the audio obtained by the microphone 2130. The cuff 2220 of the blood pressure meter 2200 expands and contracts by internal pressure being controlled, and emits exhaust sound when depressurizing. The exhaust sound of the cuff 2220 is stored in the storage unit 2170 beforehand. The audio determining unit 2113 compares the audio obtained by the microphone 2130 and the exhaust sound stored in the storage unit 2170 beforehand. The audio determining unit 2113 determines whether or not the exhaust sound emitted by the cuff 2220 is included in the audio that has been obtained by the microphone 2130, based on the comparison results, and outputs the determination results to the reliability determining unit 2114.

Note that an arrangement may be made where data where the blood pressure meter 2200 is placed at different distances and the exhaust sound of the cuff 2220 is recorded may be saved in the storage unit 2170 or an external server, and the audio determining unit 2113 compares the audio obtained by the microphone 2130 with the multiple types of exhaust sounds. This enables confirmation of the cuff 2220 being situated nearby the determining device 2100. As a result, fraudulence such as measurement by a different person, measurement with the cuff 2220 worn on the arm opposite to the side where the determining device 2100 is held, and so forth, can be eliminated.

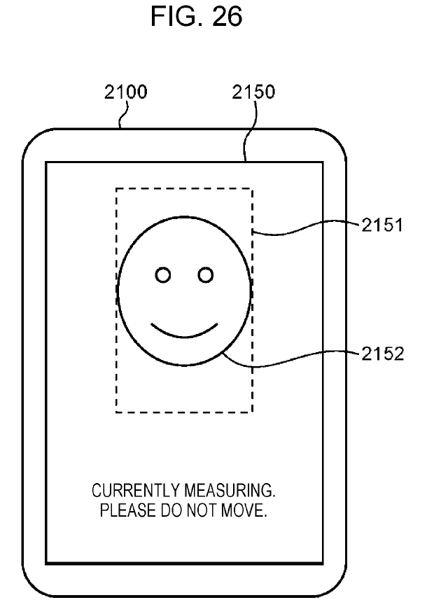

In step S2108, the facial position determining unit 2111 obtains an image including the face of the user 2000 taken by the camera 2120, obtains the second information, and notifies the obtained second information to the reliability determining unit 2114. In step S2109, the posture determining unit 2112 obtains the first information from the angle sensor 2140, and notifies the obtained first information to the reliability determining unit 2114. Thus, in the fourth embodiment, the first information and second information are obtained by the angle sensor 2140 and camera 2120 even while measuring blood pressure, and the reliability determining unit 2114 is notified. During measurement of blood pressure, the posture determining unit 2112 preferably displays a message such as “currently measuring, please do not move” on the display unit 2150 as illustrated in FIG. 26, thereby instructing the user 2000 to maintain the correct posture and not move.

When a predetermined amount of time has elapsed after starting of blood pressure measurement (step S2106), the determining device 2100 judges that blood pressure measurement has ended. Alternatively, the determining device 2100 may judge that blood pressure measurement has ended when receiving notification from the blood pressure meter 2200 via the communication unit 2160 to the effect that blood pressure measurement has ended. Thereafter, the determining device 2100 obtains the measured blood pressure measurement values from the blood pressure meter 2200 via the communication unit 2160 in step S2110.

Note that the blood pressure meter 2200 may have a speaker that emits a particular sound indicating end of measurement, for example. The audio determining unit 2113 may judge that blood pressure measurement has ended when determining that the particular sound has been emitted. The blood pressure meter 2200 may, after having emitted the particular sound from the speaker, transmit information to the determining device 2100 such as blood pressure values, time of end of measurement, and so forth.

After end of the blood pressure measurement, in steps S2111 and S2112 the reliability determining unit 2114 determines the reliability of the measurement results, using the audio data, second information, and first information, obtained respectively in steps S2107, S2108, and S2109.

Specifically, in step S2111, the reliability determining unit 2114 performs determination the same as in step S2104 regarding the first information and the second information. For example, in a case where the angle indicated by the first information is not within the predetermined first range, or in a case where the position of the face of the user 2000 indicated by the second information is not within the predetermined second range, or in a case where the proportion of size of the face of the user 2000 indicated by the second information is not within the predetermined third range (NO in step S2111), the posture has conceivably changed after starting measurement. Accordingly, the reliability determining unit 2114 determines that the reliability of measurement results is low, and returns the processing to step S2106.

Also, the reliability determining unit 2114 may continue to obtain images and angles after starting blood pressure measurement in step S2106. The reliability determining unit 2114 may measure the amount of time that the position and proportion of size of the face in the image, and the angle, were outside the predetermined ranges. In a case where this is longer than a predetermined amount of time, the reliability determining unit 2114 may determine the reliability of the measurement results to be low.

In step S2112, the reliability determining unit 2114 determines whether or not the exhaust sound of the cuff 2220 has been detected by the audio determining unit 2113 from the audio obtained in step S2107. In a case where no exhaust sound of the cuff 2220 is detected, or in a case where the detection level of the exhaust sound is at or below a predetermined threshold value (NO in step S2112), the reliability determining unit 2114 determines that the reliability of the measurement results is low, and returns the flow to step S2106.

In a case where determination is made at the reliability determining unit 2114 that the reliability of the measurement results is low (NO in step S2111 or No in step S2112), measurement is performed again by the communication unit 2160 communicating with the blood pressure meter 2200 in the example illustrated in FIG. 24, but this is not restrictive. For example, one of

(E1) discarding the measurement results as being invalid,

(E2) lowering the weighting of the measurement results when being used in analyzing, such as evaluation of health or the like, and

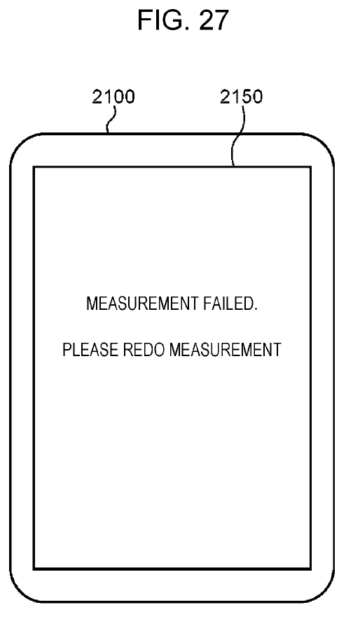

(E3) displaying a screen such as illustrated in FIG. 27 on the display unit 2150 and notifying the user 2000 that measurement has failed and prompting measurement again,

may be carried out. The measurement results of the reliability determining unit 2114 may be quantified, and the measurement results be handled differently depending on numerical values of reliability.

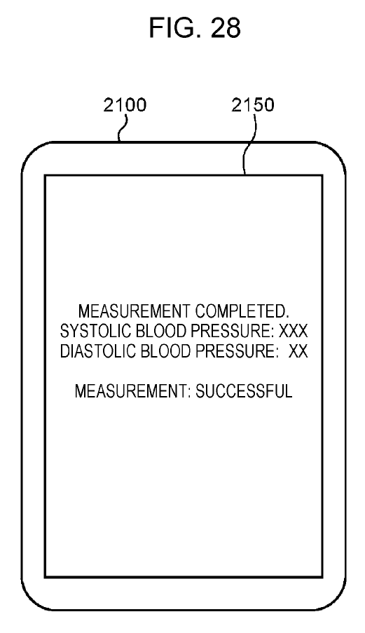

In a case where judgment is not made that reliability is low (YES in step S2111 and YES in step S2112), in step S2113 the reliability determining unit 2114 displays a screen such as illustrated in FIG. 28 on the display unit 2150, to the effect that measurement has been successful. The reliability determining unit 2114 also displays the measurement values received from the blood pressure meter 2200.

In a case where the blood pressure meter 2200 according to the fourth embodiment is not used in conjunction with the determining device 2100, the blood pressure meter 2200 may operate as a normal blood pressure meter, and may operate in cooperation with the determining device 2100 by the operation mode of the blood pressure meter 2200 having been switched.

Note that the determining device 2100 according to the fourth embodiment is for determining the posture of the user 2000, and the measurement device used is not restricted to the blood pressure meter 2200. The determining device 2100 may be used with a measurement device regarding which the posture of the user 2000 affects measurement results, such as with bathroom scales or the like. Accordingly, the reliability of measurement values can be improved.

The following advantages can be obtained by the fourth embodiment.

(1) The determining device 2100 has the camera 2120 and CPU 2110 in addition to the angle sensor 2140. Accordingly, the posture of the user 2000 can be estimated more accurately by using images of the camera 2120 in which the positional relation of the user 2000 and cuff 2220 is known, as compared to the conventional posture estimation of the user 2000 using the angle sensor alone, thereby improving accuracy of measurement results.

(2) The determining device 2100 has the camera 2120 and display unit 2150. The frame 2151 indicating the appropriate facial position is displayed as illustrated in FIG. 23, in addition to the image data taken by the camera 2120, at the display unit 2150. Accordingly, the user 2000 can easily and intuitively assume a correct posture, by feedback to the user via the display unit 2150.

(3) The determining device 2100 uses the facial image of the user 2000 taken by the camera 2120, and accordingly can detect difference in posture that the angle sensor 2140 alone cannot estimate. This also makes it more difficult for the user 2000 to perform intentionally fraudulent measurement. By eliminating readily-conceivable fraudulence, such as measuring while lying down for example, fraudulent measurement can be suppressed.

(4) The determining device 2100 has the camera 2120 and shoots the face of the user 2000 at a predetermined position, so facial recognition from the image can be easily performed. This yields advantages of easily organizing and analyzing measurement results in association with individuals regarding the measurement results, and preventing fraudulence where one person performs measurements in place of another.

### Fifth Embodiment

An example of using a wrist-worn blood pressure meter that is worn on the wrist and blood pressure is measured has been described in the fourth embodiment. An example of an upper-arm-worn blood pressure meter that is worn on the upper arm and blood pressure is measured will be described in the fifth embodiment.

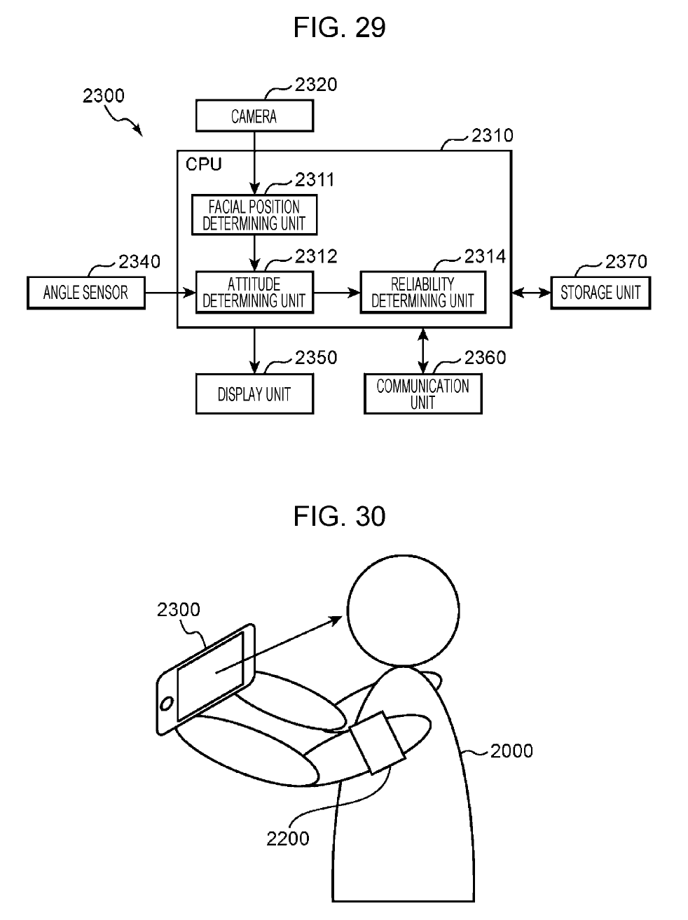

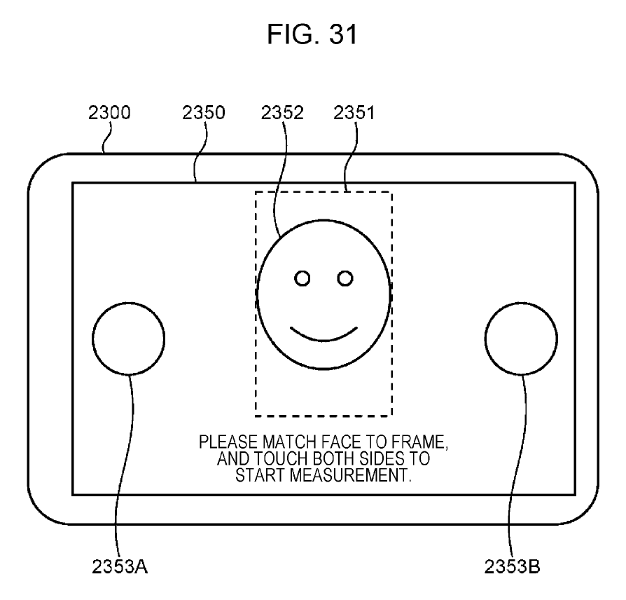

FIG. 29 is a block diagram schematically illustrating an example of the configuration of a determining device according to the fifth embodiment. FIG. 30 is a diagram schematically illustrating a measurement state when a user uses the blood pressure meter to measure blood pressure. FIG. 31 is a diagram schematically illustrating an example of a screen displayed on a display unit of the determining device before starting measurement of blood pressure. Note that the blood pressure meter used in the fifth embodiment has the same configuration as the blood pressure meter 2200 in the fourth embodiment illustrated in FIG. 21.

A determining device 2300 according to the fifth embodiment includes a camera 2320, an angle sensor 2340, a display unit 2350, a CPU 2310, a communication unit 2360, and a storage unit 2370, as illustrated in FIG. 29.

The storage unit 2370 is made up of semiconductor memory or the like, for example. The storage unit 2370 includes, for example, ROM, RAM, EEPROM, and so forth. The ROM of the storage unit 2370 stores a control program according to the fifth embodiment to operate the CPU 2310. The storage unit 2370 records information such as audio data, image data, blood pressure measurement values, and so forth. Although the storage unit 2370 is built into the determining device 2300 in the fifth embodiment, this is not restrictive. The storage unit 2370 may be memory in a server connected to a network, for example.

The CPU 2310 operates in accordance with the control program according to the fifth embodiment stored in the storage unit 2370, to function as a facial position determining unit 2311, a posture determining unit 2312, and a reliability determining unit 2314.

The determining device 2300 is held by both hands of the user 2000 who has the blood pressure meter 2200 worn on the upper arm, such that the camera 2320 takes images of the face of the user 2000, as illustrated in FIG. 30. A smartphone is used as an example of the determining device 2300 in the fifth embodiment. A tablet computer may be used as the determining device 2300 as well.

Next, the configurations of the determining device 2300 will be described. The camera 2320, angle sensor 2340, and communication unit 2360 respectively have the same functions as the camera 2120, angle sensor 2140, and communication unit 2160 in the fourth embodiment illustrated in FIG. 20.

The display unit 2350 is controlled by the CPU 2310 to display a message instructing the user 2000 to assume an appropriate posture, reliability of measurement values, and so forth. Specific examples of screens displayed on the display unit 2350 will be described later. The display unit 2350 according to the fifth embodiment is a touchscreen display, where, when the user 2000 touches the display unit 2350, the touched position is detected and notified to the CPU 2310.

The facial position determining unit 2311 has the same functions as the facial position determining unit 2111 according to the fourth embodiment illustrated in FIG. 20. The posture determining unit 2312 determines the height where the cuff 2220 is situated from the second information obtained from the facial position determining unit 2311 and the first information obtained from the angle sensor 2340. Specifically, in a case where the angle indicated by the first information is within the first range, the position of the face of the user 2000 in the image data indicated by the second information is within the second range, and the proportion of the size of the face of the user 2000 in the image data indicated by the second information is within the third range, the posture determining unit 2312 determines that the cuff 2220 and the heart of the user 2000 are at the same height. The posture determining unit 2312 outputs information to the effect that determination has been made that the cuff 2220 and the heart of the user 2000 are at the same height, to the display unit 2350 for example.

The posture determining unit 2312 may determine that the angle indicated in the first information is within the predetermined first range if the difference between the angle of inclination of the normal to the surface of the display unit 2350 of the determining device 2300 as to the gravitational direction, decided beforehand so that the position of the cuff 2220 of the blood pressure meter 2200 will be at the same height as the heart of the user 2000, and the first information obtained by the angle sensor 2340 is at or below a threshold value decided beforehand. The storage unit 2370 may store beforehand the above-described angle of inclination of the normal to the surface of the display unit 2350 of the determining device 2300 as to the gravitational direction, decided beforehand so that the position of the cuff 2220 of the blood pressure meter 2200 will be at the same height as the heart of the user 2000.

When starting measurement of blood pressure, the posture determining unit 2312 displays a frame 2351 of a size decided beforehand at a position on the display unit 2350 decided beforehand, displays icons 2353A and 2353B at positions decided beforehand at the left edge and right edge of the display unit 2350, and also displays a message to the user 2000 below the frame 2351, such as “please match face to frame, and touch both sides to start measurement”, as illustrated in FIG. 31.

The posture determining unit 2312 may determine that when a facial image 2352 of the user 2000 taken by the camera 2320 is situated within the frame 2351, and the icons 2353A and 2353B are each touched, the position of the face of the user 2000 within the image data indicated in the second information is within the predetermined second range, and the proportion of the size of the face of the user 2000 in the image data indicated by the second information is in the predetermined third range.

The size of the facial image 2352 (FIG. 31) of the user 2000 taken by the camera 2320 and displayed on the display unit 2350 is dependent on the distance between the camera 2320 (i.e., the determining device 2300), and the face of the user 2000. Accordingly, the sideways length of the frame 2351 can be decided beforehand taking into consideration a general length of the arms and size of the face of the user 2000.

The height position of the facial image 2352 of the user 2000 displayed on the display unit 2350 is dependent on the angle of the user 2000 holding the determining device 2300, and the height of the determining device 2300. Accordingly, the length and position of the frame 2351 in the vertical direction, and the angle of the normal to the surface of the display unit 2350 of the determining device 2300 as to the gravitational direction described above, can be decided beforehand so that the height of the upper arm of the user 2000 holding the determining device 2300 matches the height of the heart of the user 2000.

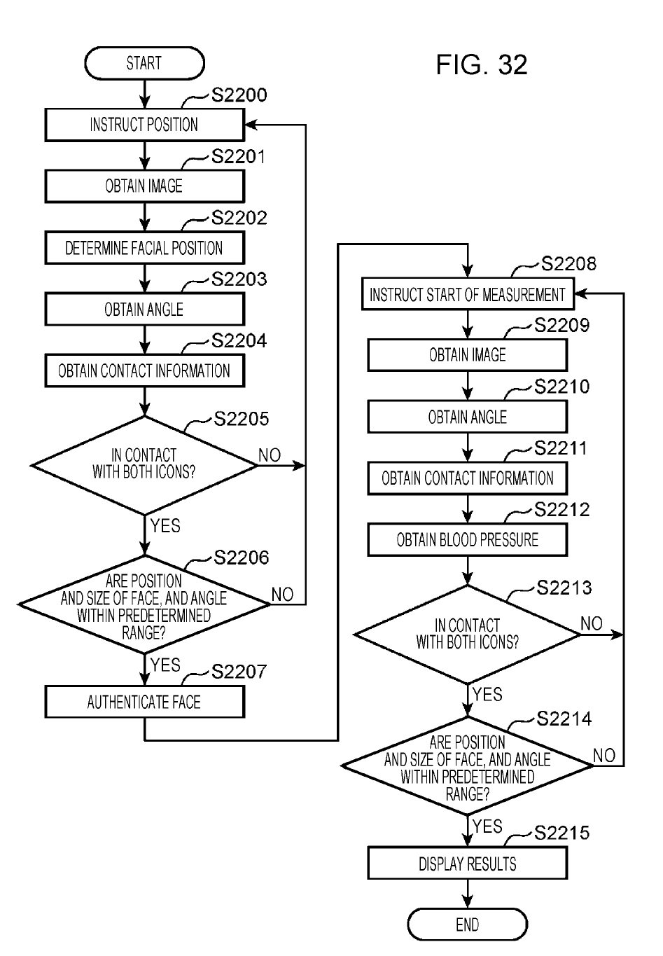

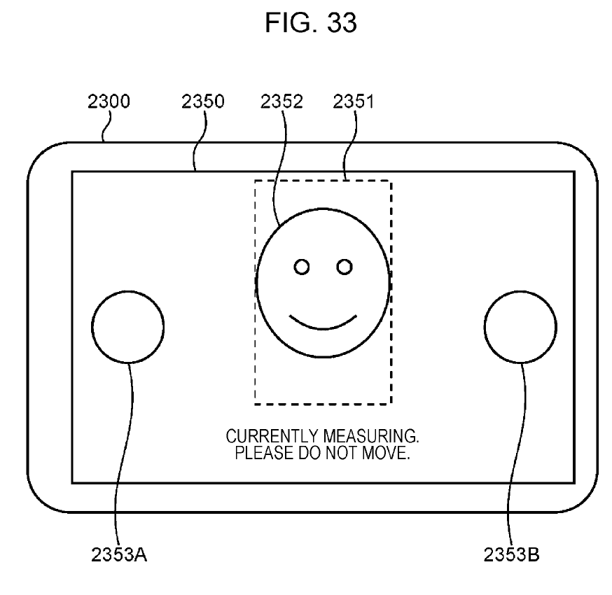

FIG. 32 is a flowchart schematically illustrating operations of the determining device 2300 when measuring blood pressure according to the fifth embodiment. FIGS. 33 through 35 are diagrams schematically illustrating examples of screens displayed on the determining device 2300 when measuring blood pressure. The operations of the determining device 2300 will be described with reference to FIGS. 31 through 35.

First, the user 2000 wears the cuff 2220 of the blood pressure meter 2200 on the upper arm. The user 2000 holds the determining device 2300 in both hands, and directs the camera 2320 toward the face of the user 2000.

In step S2200, the posture determining unit 2312 prompts the user 2000 to assume a correct posture through the display unit 2350. In this fifth embodiment, the posture determining unit 2312 displays the frame 2351 and the facial image 2352 taken by the camera 2320 on the display unit 2350, as illustrated in FIG. 31. The posture determining unit 2312 also displays the first icon 2353A near the left edge of the display unit 2350, displays the second icon 2353B near the right edge of the display unit 2350, and further displays a message “please match face to frame, and touch both sides to start measurement” below the frame 2351, thereby instructing the user 2000 to touch both the first icon 2353A and second icon 2353B.

In step S2201, the facial position determining unit 2311 obtains an image including the face of the user 2000, taken by the camera 2320. In step S2202, the facial position determining unit 2311 obtains the second information indicating the position of the face within the image and the proportion of the size of the face within the image from the obtained image, and hands the second information to the posture determining unit 2312. This position of the face is the center position of the region of the face of the user 2000 in the image data taken by the camera 2320, for example. The proportion of the size of the face is, for example, information indicating the proportion of the area of the face of the user 2000 in the image data taken by the camera 2320.

In step S2203, the posture determining unit 2312 obtains first information indicating the angle of the normal to the surface of the display unit 2350 of the determining device 2300 as to the gravitational direction, from the angle sensor 2340. In step S2204, the posture determining unit 2312 obtains contact information regarding contact to the first icon 2353A and second icon 2353B displayed on the display unit 2350, from the display unit 2350.

Based on the contact information that has been obtained, in step S2205 the posture determining unit 2312 determines whether or not the user 2000 is in contact with both of the first icon 2353A and second icon 2353B. In a case where the user 2000 is in contact with both icons (YES in step S2205), the posture determining unit 2312 judges that the positions of the hands of the user 2000 are fixed, and the flow advances to step S2206. On the other hand, in a case where the user 2000 is not in contact with both icons (NO in step S2205), the flow returns to step S2200, and the posture determining unit 2312 instructs the user 2000 to touch both of the first icon 2353A and second icon 2353B, as illustrated in FIG. 31, for example.

In step S2206, the posture determining unit 2312 determines whether the angle indicated by the first information, the position of the face of the user 2000 within the image data, and the proportion of the size of the face of the user 2000 in the image data, indicated by the second information, are each within the predetermined ranges. In a case where the angle indicated by the first information is within the first range, the position of the face of the user 2000 in the image data indicated by the second information is within the second range, and the proportion of the size of the face of the user 2000 in the image data indicated by the second information is within the third range (YES in step S2206), the posture determining unit 2312 determines that the height of the cuff 2220 and the height of the heart of the user 2000 agree, and notifies the reliability determining unit 2314 to the effect that the user 2000 is using the blood pressure meter 2200 in a correct posture.

In step S2207, the facial position determining unit 2311 uses the image obtained in step S2201 to authenticate the face of the user 2000. The storage unit 2370 stores features of the facial image of the user 2000 beforehand. The facial position determining unit 2311 extracts features of the facial image of the user 2000 from the image obtained in step S2201. The facial position determining unit 2311 compares the extracted features and the features of the facial image of the user 2000 saved in the storage unit 2370 beforehand, and determines whether matching or not. By performing this personal authentication in step S2207, the person currently using the blood pressure meter 2200 can be identified, thereby preventing blood pressure measurement by another person.

In a case where personal authentication cannot be performed in step S2207, the blood pressure measurement may be terminated. Also, in a case where determination is made by the posture determining unit 2312 that the user 2000 is at the correct posture (YES in step S2206), the flow may directly advance to step S2208.

In step S2208, the posture determining unit 2312 transmits a command to the blood pressure meter 2200 to start measurement, via the communication unit 2360.

In step S2209, the facial position determining unit 2311 obtains an image including the face of the user 2000 taken by the camera 2320, obtains the second information, and notifies the obtained second information to the reliability determining unit 2314. In step S2210, the posture determining unit 2312 obtains the first information from the angle sensor 2340, and notifies the obtained first information to the reliability determining unit 2314. In step S2211, the posture determining unit 2312 obtains contact information regarding the first icon 2353A and second icon 2353B displayed on the display unit 2350, from the display unit 2350.

Thus, in the fifth embodiment, the first information and second information are obtained by the angle sensor 2340 and camera 2320 even while measuring blood pressure, contact information regarding the first icon 2353A and second icon 2353B is obtained from the display unit 2350, and the reliability determining unit 2314 is notified of the obtained information. During measurement of blood pressure, the posture determining unit 2312 may display a message such as “currently measuring, please do not move” on the display unit 2350 as illustrated in FIG. 33, thereby instructing the user 2000 to maintain the correct posture and not move.

When a predetermined amount of time has elapsed after starting of blood pressure measurement (step S2208), the determining device 2300 judges that blood pressure measurement has ended. Alternatively, the determining device 2300 may judge that blood pressure measurement has ended when receiving notification from the blood pressure meter 2200 via the communication unit 2360 to the effect that the blood pressure measurement has ended. Thereafter, the determining device 2300 obtains the measured blood pressure measurement values from the blood pressure meter 2200 via the communication unit 2360 in step S2212.

After end of the blood pressure measurement, in steps S2213 and S2214 the reliability determining unit 2314 determines the reliability of the measurement results, using the image data, angle data, and contact information, obtained respectively in steps S2209, S2210, and S2211, using the same procedures as in step S2205 and S2206.

Note that a microphone and an audio determining unit may be provided in the same way as in the fourth embodiment, to perform determination by audio. This can further improve reliability.

In a case where determination is made at the reliability determining unit 2314 that the reliability of the measurement results is low (NO in step S2213 or NO in step S2214), measurement is performed again by the communication unit 2360 communicating with the blood pressure meter 2200 in the example illustrated in FIG. 32, but this is not restrictive. For example, one of

(F1) discarding the measurement results as being invalid,

(F2) lowering the weighting of the measurement results when being used in analyzing, such as evaluation of health or the like, and

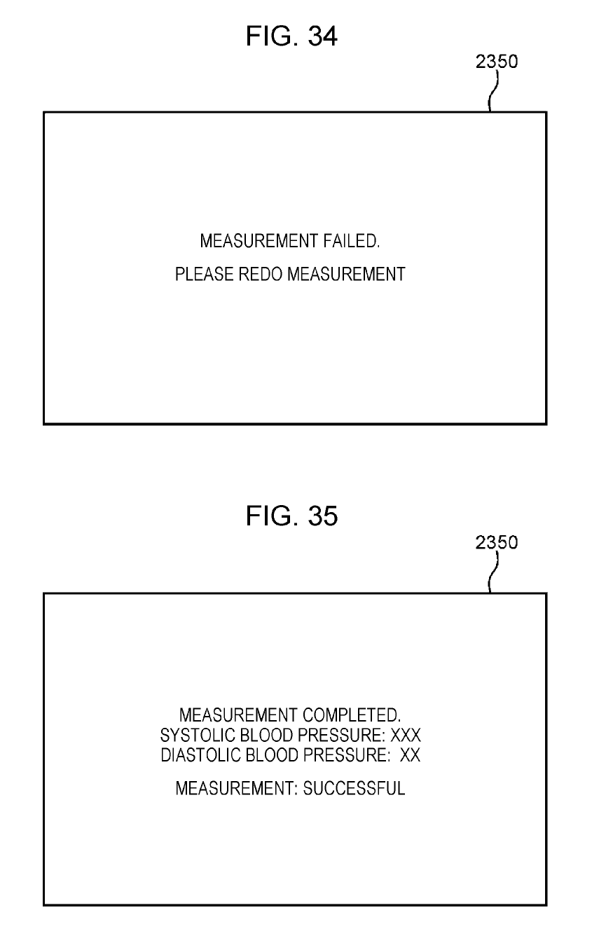

(F3) displaying a screen such as illustrated in FIG. 34 on the display unit 2350 and notifying the user 2000 that measurement has failed and prompting measurement again,

may be carried out. The measurement results of the reliability determining unit 2314 may be quantified, and the measurement results may be handled differently depending on numerical values of reliability.

In a case where judgment is not made that reliability is low (YES in step S2213 and YES in step S2214), in step S2215 the reliability determining unit 2314 displays a screen such as illustrated in FIG. 35 on the display unit 2350, to the effect that measurement has been successful. The reliability determining unit 2314 also displays the measurement values received from the blood pressure meter 2200.

According to the fifth embodiment, in addition to the advantages of the fourth embodiment described above, the positions of both hands of the user 2000 are fixed by using contact information of contact with the first icon 2353A and second icon 2353B displayed on the display unit 2350, thereby enabling accurate posture determination.

### Sixth Embodiment

A technique for identifying a measurement subject with a determining device that determines a measurement state when measuring blood pressure using a blood pressure meter, will be described in a sixth embodiment. The technique for identifying a measurement subject according to the sixth embodiment is different from step S2105 in FIG. 24 and step S2207 in FIG. 32.

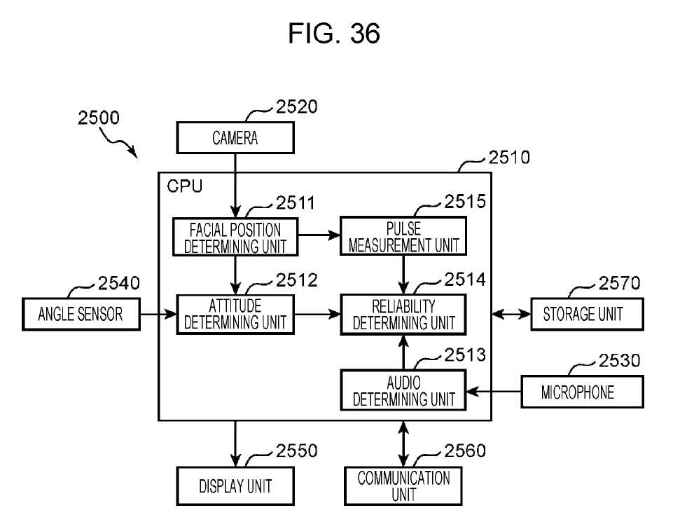

FIG. 36 is a block diagram schematically illustrating a configuration example of a determining device according to the sixth embodiment. The measurement state at the time of the user measuring blood pressure using the blood pressure meter in the sixth embodiment is the same as in the fourth embodiment illustrated in FIG. 22. The screen displayed on the display unit of the determining device before start measuring blood pressure in the sixth embodiment is the same as with the fourth embodiment illustrated in FIG. 23.

The blood pressure meter used in the sixth embodiment has the same configuration as with the fourth embodiment illustrated in FIG. 21. Note however, the blood pressure meter 2200 used in the sixth embodiment (FIG. 21) measures pulse, in addition to blood pressure as with a normal blood pressure meter. The control unit 2210 of the blood pressure meter 2200 transmits the results of measuring pulse to a determining device 2500 via the communication unit 2230, in addition to the measurement results of blood pressure.

The determining device 2500 according to the sixth embodiment has a CPU 2510, a camera 2520, a microphone 2530, an angle sensor 2540, a display unit 2550, a communication unit 2560, and a storage unit 2570, as illustrated in FIG. 36.

The storage unit 2570 is made up of semiconductor memory or the like, for example. The storage unit 2570 includes, for example, ROM, RAM, EEPROM, and so forth. The ROM of the storage unit 2570 stores a control program according to the sixth embodiment to operate the CPU 2510. The storage unit 2570 records information such as audio data, image data, blood pressure measurement values, and so forth. Although the storage unit 2570 is built into the determining device 2500 in the sixth embodiment, this is not restrictive. The storage unit 2570 may be memory in a server connected to a network, for example.

The CPU 2510 operates in accordance with the control program according to the sixth embodiment stored in the storage unit 2570, to function as a facial position determining unit 2511, a posture determining unit 2512, an audio determining unit 2513, a reliability determining unit 2514, and a pulse measurement unit 2515.

In FIG. 36, the camera 2520, microphone 2530, angle sensor 2540, display unit 2550, and communication unit 2560 respectively function the same as the camera 2120, microphone 2130, angle sensor 2140, display unit 2150, and communication unit 2160 of the fourth embodiment illustrated in FIG. 20. The facial position determining unit 2511, posture determining unit 2512, and audio determining unit 2513 respectively function the same as the facial position determining unit 2111, posture determining unit 2112, and audio determining unit 2113 of the fourth embodiment illustrated in FIG. 20.

The pulse measurement unit 2515 measures the pulse of the user 2000 by analyzing the coloration information of the body surface from the moving image including the face of the user 2000, that has been taken by the camera 2520. The pulse measurement unit 2515 measures the pulse of the user 2000 by the procedures illustrated in FIG. 10, for example.

The reliability determining unit 2514 functions the same as the reliability determining unit 2114 according to the fourth embodiment (FIG. 20). The reliability determining unit 2514 further receives the measurement results of pulse transmitted from the blood pressure meter 2200 (FIG. 3) via the communication unit 2560. The reliability determining unit 2514 compares the pulse of the user 2000 measured by the pulse measurement unit 2515 with the pulse transmitted from the blood pressure meter 2200, and determines whether the intervals of pulse waves between the two match. Upon determining that these match, the reliability determining unit 2514 continues the blood pressure measurement operations described in the fourth embodiment, for example, as normal processing. If determining that these do not match, the reliability determining unit 2514 terminates the blood pressure measurement operations described in the fourth embodiment, for example, as abnormality processing.

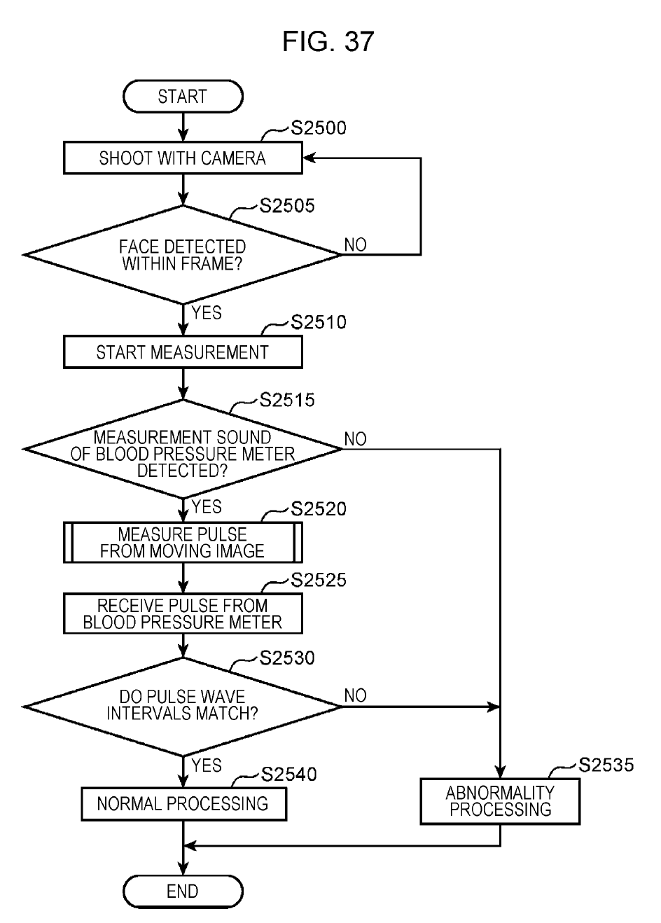

FIG. 37 is a flowchart schematically illustrating the operations of the determining device 2500 identifying the measurement subject, in the sixth embodiment. In step S2500, the camera 2520 shoots the user 2000. The facial position determining unit 2511 obtains the image of the user 2000 shot by the camera 2520. In step S2505, the facial position determining unit 2511 extracts a facial image of the user 2000 from the obtained image. The facial position determining unit 2511 notifies the position of the extracted facial image to the posture determining unit 2512.

The posture determining unit 2512 determines whether or not the facial image at the position that has been notified (e.g., facial image 2152 in FIG. 23) has been detected within a frame (e.g., frame 2151 in FIG. 23) displayed on the display unit 2550. If the position of the facial image is within the frame (YES in step S2505), the processing advances to step S2510. On the other hand, if the position of the facial image is not within the frame (NO in step S2505), the processing returns to step S2500, and the above-described processing is repeated.

In step S2510, the posture determining unit 2512 instructs the blood pressure meter 2200 to start blood pressure measurement. In step S2515, the audio determining unit 2513 determines whether or not the exhaust sound of the cuff 2220 of the blood pressure meter 2200 has been detected from the audio obtained by the microphone 2530. In a case where the exhaust sound of the cuff 2220 is detected (YES in step S2515), the flow advances to step S2520. On the other hand, in a case where no exhaust sound of the cuff 2220 is detected (NO in step S2515), the flow advances to step S2535.

In step S2520, the pulse measurement unit 2515 measures the pulse of the user 2000 by analyzing in time sequence the coloration information of the body surface from the moving image including the face of the user 2000, taken by the camera 2520 and extracted by the facial position determining unit 2511. In step S2520, the procedures illustrated in FIG. 10, for example, are executed, and the pulse of the user 2000 is measured. The pulse measurement unit 2515 notifies the measured pulse to the reliability determining unit 2514.

In step S2525, the reliability determining unit 2514 receives the pulse from the blood pressure meter 2200 via the communication unit 2560. In step S2530, the reliability determining unit 2514 compares the pulse waves notified from the pulse measurement unit 2515 with the pulse waves of the pulse received from the blood pressure meter 2200, and determines whether the intervals of the two match. If the intervals between pulse waves of the pulse notified from the pulse measurement unit 2515 and intervals between pulse waves of the pulse received from the blood pressure meter 2200 match (YES in step S2530), the flow advances to step S2540. If the intervals between pulse waves of the pulse notified from the pulse measurement unit 2515 and intervals between pulse waves of the pulse received from the blood pressure meter 2200 do not match (NO in step S2530), the flow advances to step S2535.

In step S2535, the reliability determining unit 2514 terminates blood pressure measurement, as abnormality processing. In step S2540, the reliability determining unit 2514 continues blood pressure measurement as normal processing. Processing of step S2106 and thereafter in FIG. 24, for example, may be performed in step S2540.

As described above, in the sixth embodiment, the pulse measurement unit 2515 measures the pulse of the user 2000 by analyzing, in time sequence, the coloration information of the body surface from the moving image including the face of the user 2000 taken by the camera 2520, and notifies the measurement results to the reliability determining unit 2514. The reliability determining unit 2514 also receives pulse measured by the blood pressure meter 2200 via the communication unit 2560. The reliability determining unit 2514 determines whether or not the intervals of pulse waves of the pulse of the user 2000 and the pulse measured by the blood pressure meter 2200 match. Thus, according to the sixth embodiment, the user 2000 being taken by the camera 2520 and the measurement subject of whose blood pressure is being measured by the blood pressure meter 2200 can be determined to be the same, in a sure manner. Although the determining device 2500 (FIG. 36) is described as being used in the sixth embodiment, the determining device 2100 (FIG. 20) according to the fourth embodiment or the determining device 2300 (FIG. 29) according to the fifth embodiment may be used instead.

### Modifications

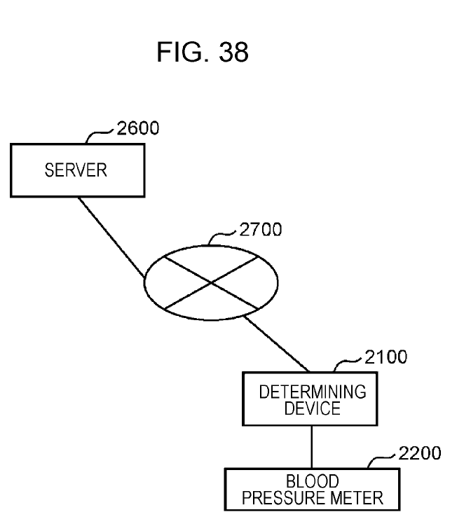

FIG. 38 is a block diagram schematically illustrating an example of the configuration of a system having the determining device and a server. The determining device 2100 and a server 2600 are connected to a network 2700, and are configured to be capable of communication with each other, as illustrated in FIG. 38. The server 2600 is installed externally, such as at a medical institution, an insurance provider, or the like. Although the determining device 2100 (FIG. 20) according to the fourth embodiment is illustrated as being connected to the server 2600 in the example in FIG. 38, the determining device 2300 (FIG. 29) according to the fifth embodiment or the determining device 2500 (FIG. 36) according to the sixth embodiment may be connected to the server 2600.

In step S2113 in FIG. 24, the reliability determining unit 2114 may further notify the blood pressure measurement results and the determination results of the reliability determining unit 2114 to an external device such as the server 2600 or the like, a medical institution, or an insurance provider, via the communication unit 2160. At this time, whether or not to transmit, and information to be transmitted, may be changed in accordance with the determination results by the reliability determining unit 2114. In a case where personal authentication has been performed, the reliability determining unit 2114 preferably transmits with personal identification information and measurement results associated.

Also, in step S2215 in FIG. 32, the reliability determining unit 2314 may further transmit the blood pressure measurement results and determination results of the reliability determining unit 2314 to an external device such as the server 2600 or the like, a medical institution, or an insurance provider, via the communication unit 2360. At this time, whether or not to transmit, and information to be transmitted, may be changed in accordance with the determination results by the reliability determining unit 2314. In a case where personal authentication has been performed, the reliability determining unit 2314 preferably transmits with personal identification information and measurement results associated.

The technology realized by the present disclosure, whereby personal authentication information is succeeded among multiple measurement devices by comparing biometric information enables measurement data of measurement devices that do not have personal authentication functions to be collected at a server and saved in a format associated with an individual, which is advantageous in accurately comprehending and estimating the health state of the user. The determining device according to the present disclosure is also advantageous in improvement of reliability of household use blood pressure meters, for example.

What is claimed is:

1. A blood pressure measurement state determination method for determining a blood pressure measurement state, using a determining device that is held in a hand of a user to whom a blood pressure meter is mounted, the method comprising:
obtaining image data including a face of the user by a camera that the determining device has;
obtaining first information indicating an inclination angle of the determining device as to the gravitational direction, by an angle sensor that the determining device has;
obtaining second information indicating a position of the face of the user in the image data, and the proportion of the size of the face of the user in the image data;
determining whether or not the angle indicated in the first information is within a first range;
determining whether or not the position of the face of the user indicated in the second information is within a second range;
determining whether or not the proportion of the size of the face of the user indicated in the second information is within a third range;
determining whether or not the user is correctly using the blood pressure meter;
providing a first notification indicating that the blood pressure meter is being correctly used when it is determined that the user is correctly using the blood pressure meter;
providing a second notification indicating the blood pressure meter is not correctly using when it is determined that the user is not correctly using the blood pressure meter,
wherein in the determination regarding whether or not the user is correctly using the blood pressure meter, determination is made that the user is correctly using the blood pressure meter when the angle indicated in the first information is determined to be within the first range, the position of the face of the user indicated in the second information is determined to be within the second range, and the proportion of the size of the face of the user indicated in the second information is determined to be within the third range.

2. The blood pressure measurement state determination method according to claim 1, further comprising:
obtaining audio around the determining device; and
determining whether or not the obtained audio and audio emitted by the blood pressure meter, stored in a storage unit beforehand, agree,
wherein, the determining of whether or not the user is correctly using the blood pressure meter further includes determining that the user is using the blood pressure meter correctly if determination is made that the obtained audio and audio emitted by the blood pressure meter stored in the storage unit agree.

3. The blood pressure measurement state determination method according to claim 1, further comprising:
transmitting a command instructing to start measurement of blood pressure of the user to the blood pressure meter upon determination being made that the user is using the blood pressure meter correctly.

4. The blood pressure measurement state determination method according to claim 3, further comprising:
determining again whether or not the user is using the blood pressure meter correctly, after a predetermined amount of time has elapsed after transmitting the command.

5. The blood pressure measurement state determination method according to claim 3, further comprising:
determining again whether or not the user is using the blood pressure meter correctly, upon receiving a notification is received from the blood pressure meter indicating that blood pressure measurement of the user has ended.

6. The blood pressure measurement state determination method according to claim 1, further comprising:
displaying a first icon indicating the position to hold the determining device with one hand out of the left hand and right hand of the user, and a second icon indicating the position to hold the determining device with other hand out of the left hand and right hand of the user, on a touch panel display unit provided to the determining device,
wherein the determining of whether or not the user is correctly using the blood pressure meter further includes determining that the user is using the blood pressure meter correctly if detecting that the one hand of the user is in contact with the first icon and that the other hand of the user is in contact with the second icon.

7. The blood pressure measurement state determination method according to claim 1, further comprising:
determining whether or not the image data matches image data of the user of the determining device stored in the storage unit beforehand,
wherein the determining of whether or not the user is correctly using the blood pressure meter further includes determining that the user is using the blood pressure meter correctly if detecting that the image data matches image data of the user of the determining device stored in the storage unit.

8. The blood pressure measurement state determination method according to claim 1, wherein the first notification and the second notification are provided to a server connected to the determining device.

9. The blood pressure measurement state determination method according to claim 1, wherein the first notification and the second notification are provided to the user.

10. The blood pressure measurement state determination method according to claim 9, further comprising:
prompting the user to use the blood pressure meter correctly when it is determined that the user is not correctly using the blood pressure meter.

11. A blood pressure measurement state determining device for determining a blood pressure measurement state device that is held in a hand of a user to whom a blood pressure meter is mounted, the determining device comprising:
a camera that obtains image data including a face of the user;
an angle sensor that obtains first information indicating an inclination angle of the determining device as to the gravitational direction;
a processor; and
and a display,
wherein the processor
obtains second information indicating a position of the face of the user in the image data, and the proportion of the size of the face of the user in the image data,
determines whether or not the angle indicated in the first information is within a first range, determines whether or not the position of the face of the user indicated in the second information is within a second range, and determines whether or not the proportion of the size of the face of the user indicated in the second information is within a third range, and
determines that the user is correctly using the blood pressure meter if determination is made that the angle indicated in the first information is within a first range, determination is made that the position of the face of the user indicated in the second information is within a second range, and determination is made that the proportion of the size of the face of the user indicated in the second information is within a third range,
and wherein the display
displays a notification indicating to the user that the blood pressure meter is being correctly used upon determination being made that the user is correctly using the blood pressure meter, and
displays a notification prompting the user to use the blood pressure meter correctly upon determination being made that the user is not correctly using the blood pressure meter.

12. A non-transitory computer-readable recording medium storing a program for determining a blood pressure measurement state, using a determining device that determines a blood pressure measurement state and is held in a hand of a user to whom a blood pressure meter is mounted,
the program causing a computer of the determining device to execute
obtaining image data including a face of the user by a camera that the determining device has,
obtaining first information indicating an inclination angle of the determining device as to the gravitational direction, by an angle sensor that the determining device has,
obtaining second information indicating a position of the face of the user in the image data, and the proportion of the size of the face of the user in the image data,
determining whether or not the angle indicated in the first information is within a first range,
determining whether or not the position of the face of the user indicated in the second information is within a second range,
determining whether or not the proportion of the size of the face of the user indicated in the second information is within a third range, determining whether or not the user is correctly using the blood pressure meter,
providing a first notification indicating that the blood pressure meter is being correctly used when it is determined that the user is correctly using the blood pressure meter, and
providing a second notification indicating that the blood pressure meter is not correctly used when it is determined that the user is not correctly using the blood pressure meter,
wherein in the determination regarding whether or not the user is correctly using the blood pressure meter, determination is made that the user is correctly using the blood pressure meter when the angle indicated in the first information is determined to be within the first range, the position of the face of the user indicated in the second information is determined to be within the second range, and the proportion of the size of the face of the user indicated in the second information is determined to be within the third range.
